(19) United States
(12) Patent Application Publication
(10) Pub. No.: US 2025/0277226 A1
(43) Pub. Date: Sep. 4, 2025

(54) IMPROVED BIOTECHNOLOGICAL METHOD FOR PRODUCING GUANIDINO ACETIC ACID (GAA) BY INACTIVATION OF AN AMINO ACID EXPORTER

(71) Applicant: Evonik Operations GmbH, Essen (DE)

(72) Inventors: Frank Jankowitsch, Sassenberg (DE); Kay Marin, Borgholzhausen (DE); Frank Schneider, Halle (DE); Brigitte Bathe, Salzkotten (DE)

(73) Assignee: Evonik Operations GmbH, Essen (DE)

(21) Appl. No.: 18/562,157

(22) PCT Filed: May 10, 2022
(86) PCT No.: PCT/EP2022/062663

§ 371 Date: Nov. 17, 2023

(30) Foreign Application Priority Data
May 21, 2021 (EP) 21175138.3
Nov. 16, 2021 (EP) 21208485.9

(51) Int. Cl. C12N 15/77; C07K 14/245; C07K 14/34; C12N 9/00; C12N 9/06; C12N 9/10; C12N 9/88; C12N 15/70; C12P 13/04; C12P 13/10; C12R 1/15; C12R 1/19

(52) U.S. Cl. CPC C12N 15/77; C07K 14/245; C07K 14/34; C12N 9/0016; C12N 9/1007; C12N 9/1018; C12N 9/1051; C12N 9/88; C12N 9/93; C12N 15/70; C12P 13/04; C12P 13/10; C12R 2001/15; C12R 2001/19; C12Y 104/01002; C12Y 201/01002; C12Y 201/03003; C12Y 201/04001; C12Y 403/02001; C12Y 603/04005; C12Y 603/04016

(57) ABSTRACT

A microorganism is transformed to be capable of producing guanidinoacetic acid (GAA) having an inactivated amino acid exporter. The microorganism is used in a method for the fermentative production of GAA. Moreover, creatine is produced by a method of fermentative production.

### CROSS-REFERENCE TO RELATED APPLICATIONS

This application is a National Stage entry under § 371 of International Application No. PCT/EP20221062663, filed on May 10, 2022, and which claims the benefit of priority to European Patent Application No. 21175138.3, filed on May 21, 2021, and European Patent Application No. 21208485.9, filed on Nov. 16, 2021. The content of each of these applications is hereby incorporated by reference in its entirety.

### REFERENCE TO A SEQUENCE LISTING

The present invention is accompanied by an ASCII text file as a computer readable form containing the sequence listing entitled, “2024-09-30-005779USPCT-Sequence-List-as-filed.txt”, created on Sep. 30, 2024, with a file size of 55,912 bytes, the content of which is hereby incorporated by reference in its entirety.

### BACKGROUND OF THE INVENTION

### Field of the Invention

The present invention relates to a microorganism transformed to be capable of producing guanidinoacetic acid (GAA) and to a method for the fermentative production of GAA using such microorganism. The present invention also relates to a method for the fermentative production of creatine.

### Description of Related Art

GAA is an organic compound used as animal feed additive (WO2005120246 A1/US2011257075 A1). GAA is a natural precursor of creatine. Therefore, the supplementation of GAA allows for an optimal supply of creatine in the organism.

The present invention pertains to a method to produce GAA by a fermentative process using industrial feed stocks (e.g. ammonia, ammonium salts and glucose or sugar containing substrates) as starting material. In biological systems GAA and ornithine are formed from arginine and glycine as starting materials by the catalytic action of an L-arginine:glycine-amidinotransferase (AGAT; EC 2.1.4.1), which is the first step in creatine biosynthesis:

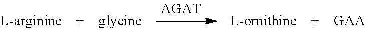

Guthmiller et al. (J Biol Chem. 1994 Jul. 1; 269(26):17556-60) have characterized a rat kidney AGAT by cloning and heterologously expressing the enzyme in E. coli. Muenchhoff et al. (FEBS Journal 277 (2010) 3844-3860) report the first characterization of an AGAT from a prokaryote also by cloning and heterologously expressing the enzyme in E. coli. For the production of GAA from L-arginine and glycine by a whole cell catalyst Zhang et al. have designed a reconstituted ornithine cycle in Escherichia coli by introducing a heterogenous AGAT from different species (e g. Homo sapiens, Cylindrospermopsis raciborskii, Moorea producens) and by introducing a citrulline synthesis module (e.g. ovexpression of carAB, argF and argI) and an arginine synthesis module (e.g. overexpression of argG, argH; introduction of aspA) into Escherichia coli(Yiwen Zhang, Hang Zhou, Yong Tao, and Baixue Lin, ACS Synth. Biol. 2020, 9, 2066-2075).

Several approaches for increasing the production of one of the starting materials in GAA synthesis, i.e. L-arginine, in microorganisms, particularly bacteria, are also known from literature. An overview for the metabolic engineering of Corynebacterium glutamicum (C glutamicum) for L-arginine production is provided by Park et al. (NATURE COMMUNICATIONS|DOI: 10.1038/ncomms5618) They propose random mutagenesis and screening for L-arginine producers of already L-arginine producing C. glutamicum strains, e.g. of ATCC 21831 (Nakayama and Yoshida 1974, U.S. Pat. No. 3,849,250 A) and stepwise rational metabolic engineering based on system-wide analysis of metabolism results in a gradual increase in L-arginine production throughout the strain engineering steps. Yim et al. (J Ind Microbiol Biotechnol (2011) 38.1911-1920) could show that inactivation of the argR, gene coding for the central repressor protein ArgR controlling the L-arginine biosynthetic pathway, by disrupting the chromosomal argR gene in C. glutamicum leads to an improved arginine-producing strain. Ginesy et al. (Microbial Cell Factories (2015) 14.29) report the successful engineering of E. coli for enhanced arginine production Among other, they proposed the deletion of the argR repressor gene.

Kurahashi et al. (EP1057893 A1) report on methods for increasing the L-arginine producing ability of a microorganism by enhancing L-arginine biosynthesis enzymes utilizing recombinant DNA techniques, e.g. by utilizing a microorganism belonging to the genus Corynebacterium or Brevibacterium which is made to harbor a recombinant DNA comprising a vector DNA and a DNA fragment containing genes for acetylornithine deacetylase, N-acetylglutamic acid-γ-semialdehyde dehydrogenase, N-acetyl glutamokinase and argininosuccinase derived from a microorganism belonging to the genus Escherichia. For an improved L-arginine production the authors further propose a microorganism which is enhanced in an activity of intracellular glutamate dehydrogenase (GDH) and which has an L-arginine producing ability.

A method of using a genetic recombinant strain, wherein a gene which inhibits the expression of arginine-biosynthesizing operon argR was inactivated has been reported by Suga et al. (U.S. Pat. No. 7,160,705 B2). In particular, the deletion in argR, which controls the arginine operon, has been considered as an important factor in arginine production. In a microorganism of Corynebacterium, the argCJBDFR gene, which is involved in arginine biosynthesis, is constituted in the form of an operon and is subjected to feedback-inhibition by intracellular arginine (Sakanyan et al., Microbiology, 142:9-108, 1996), thus imposing a limitation on its high yield L-arginine production. The arginine operon is an operon consisting of genes encoding enzymes involved in the mechanism of L-arginine biosynthesis, and in particular, arginine operon consists of genes encoding enzymes constituting the cyclic steps of L-arginine biosynthesis. Specifically, the arginine operon consists of N-acetylglutamyl phosphate reductase (ArgC), glutamate N-acetyltransferase (ArgJ), N-acetylglutamate kinase (ArgB), acetylomithine aminotransferase (ArgD), ornithine carbamoyltransferase (ArgF), and the arginine repressor (ArgR). These enzymes are involved in the continuous enzyme reactions of L-arginine biosynthesis.

According to the literature, the amino acid exporter LysE catalyzes the cellular export of L-lysine, but also of L-arginine and L-citrulline. LysG activates the transcription of the gene lysE, which encodes LysE the amino acid exporter. LysG requires a co-inducer such as L-lysine, L-arginine, L-citrulline or L-histidine (Lubitz et al. (2016). “Roles of export genes cgmA and lysE for the production of L-arginine and L-citrulline by Corynebacterium glutamicum.” Appl Microbiol Biotechnol 100(19): 8465-8474). Lubitz et al. use the C. glutamicum strain ARG2 (Peters-Wendisch et al (2014) Engineering biotin prototrophic Corynebacterium glutamicum strains for amino acid, diamine and carotenoid production. J Biotechnol. doi:10.1016/j.jbiotec.2014.01.023) which is characterized by the inactivation of the argR gene by deletion in combination with the plasmid-based expression of a feedback-resistant allele of ArgB (ArgB^{fbr}). For this strain carrying both modifications the authors describe the accumulation of L-arginine in the supernatant of the cultures. Furthermore, Lubitz et al describe that in such a strain the inactivation of the genes lysE and cmg, encoding for membrane proteins, results in a reduction of the arginine formation.

Ginesy et al. (M. Ginesy et al., Microbiol Cell Factories (2015) 14:29, DOI 10.1186/s12934-015-0211-y) could show that arginine production with an E. coli producing strain having among other a deleted argR repressor gene can be increased by overexpression of the arginine exporter system.

However, in order to obtain relatively high L-arginine concentrations in the cell it is necessary to prevent L-arginine exportation. The amino acid exporter LysE counteracts the intracellular arginine concentration and reduces the substrate availability by efficiently transporting the substrate arginine from the cell. In addition, the citrulline from arginine biosynthesis is also secreted into the medium by an active LysE exporter. LysE is regulated by the transcriptional activator LysG (Bellmann, A., et al. (2001). “Expression control and specificity of the basic amino acid exporter LysE of Corynebactenum glutamicum.” Microbiology (Reading) 147 (Pt 7): 1765-1774).

Fan Wenchao discloses a method for the production of creatine by fermentation of non-pathogenic microorganisms, such as Corynebacterium glutamicum (CN106065411 A). The microorganism has the following biotransformation functions: glucose conversion to L-glutamic acid; conversion of L-glutamic acid to N-acetyl-L-glutamic acid; conversion of N-acetyl-L-glutamic acid to N-acetyl-L-glutamic acid semialdehyde; conversion of N-acetyl-L-glutamic acid semialdehyde to N-acetyl-L-ornithine; conversion of N-acetyl-L-ornithine to L-ornithine; conversion of L-omithine to L-citrulline; conversion of L-citrulline to arginino-succinic acid; conversion of arginino-succinic acid to L-arginine; conversion of L-arginine to guanidinoacetic acid; and, finally, conversion of guanidinoacetic acid to creatine Fan Wenchao proposes, that the microorganism overexpresses one or more enzymes selected from the group consisting of N-acetylglutamate-synthase, N-acetylomithine-δ-aminotransferase, N-acetylomithinase, omithine-carbamoyl transferase, argininosuccinate synthetase, glycine amidino-transferase (EC: 2.1. 4.1), and guanidinoacetate N-methyltransferase (EC: 2.1.1.2). The microorganism overexpresses preferably glycine aminotransferase (L-arginine:glycine amidinotransferase) and guanidinoacetate N-methyltransferase.

### SUMMARY OF THE INVENTION

The problem underlying the present invention is to provide an improved microorganism transformed to be capable of producing guanidinoacetic acid (GAA) and to a method for the fermentative production of GAA using such microorganism.

### DETAILED DESCRIPTION OF THE INVENTION

The problem is solved by a microorganism having an increased ability to provide L-arginine compared with the ability of the wildtype microorganism and comprising at least one gene coding for a protein having the function of an L-arginine:glycine amidinotransferase and having a decreased activity of a protein having the function of an arginine exporter compared with the activity of the respective protein in the wildtype microorganism at the same status of the cell cycle.

This means that the protein having the function of an arginine exporter in the microorganism according to the present invention shows at any time and circumstances during the cell cycle an artificially designed decreased activity compared with the activity of the respective protein in the wildtype microorganism at the same time and circumstances during the cell cycle.

The microorganism according to the present invention is preferably a genetically modified organism (GMO) that does not naturally occur. In a GMO the genetic material has been altered using genetic engineering techniques. Preferably, at least one gene coding for a protein having the function of an L-arginine:glycine amidinotransferase has been introduced using genetic engineering techniques. Preferably, also the decreased activity of the protein having the function of an arginine exporter compared with the activity of the respective protein in the wildtype microorganism has been achieved using genetic engineering techniques.

Proteins having the function of an L-arginine:glycine amidinotransferase (AGAT) belong to the amidinotransferase family. The amidinotransferase family comprises glycine (EC:2.1.4.1) and inosamine (EC.2.1.4.2) amidinotransferases, enzymes involved in creatine and streptomycin biosynthesis respectively. This family also includes arginine deiminases, EC:3.5.3.6. These enzymes catalyse the reaction: arginine+H₂O <=>citrulline+NH₃. Also found in this family is the Streptococcus anti tumour glycoprotein. Enzymes or proteins with an L-arginie:glycine-amidinotransferase (AGAT) activity are also described to possess a conserved domain that belongs to the PFAM Family: Amidinotransf (PF02274) (Marchler-Bauer A et al. (2017), “CDD/SPARCLE: functional classification of proteins via subfamily domain architectures.”, Nucleic Acids Res. 45 (D1):D200-D203.) as described also in the following publications: Pissowotzki K et al., Mol Gen Genet 1991; 231:113-123 (PUBMED:1661369 EPMC:1661369); D'Hooghe I et al., J Bacteriol 1997; 179:7403-7409 (PUBMED:9393705 EPMC:9393705); Kanaoka M et al., Jpn J Cancer Res 1987; 78:1409-1414 (PUBMED.3123442 EPMC.3123442). Particular examples of AGATs are those of Moorea producens, Homo sapiens, Rattus norvegicus, Galeopterus variegatus, and of Cylindrospermopsis raciborskii.

In the context of the present invention, a microorganism having an improved ability to provide L-arginine means a microorganism producing or recycling L-arginine in excess of its own need. This property may be achieved by selection of microorganisms that are natural L-arginine producers or may have acquired the ability to produce L-arginine by mutation. Examples for such L-arginine producing microorganisms are e.g. C. glutamicum ATCC 21831 or those disclosed by Park et al. (NATURE COMMUNICATIONS I DOI: 10.1038/ncomms5618) or by Ginesy et al. (Microbial Cell Factories (2015) 14:29).

In one embodiment of the present invention, the argR gene coding for the arginine responsive repressor protein ArgR in the microorganism according to the present invention is attenuated or deleted.

The activity of an enzyme having the function of a carbamoyl phosphate synthase (EC 6.3.4.16, e.g. CarAB) in the microorganism according to the present invention may be increased compared to the respective enzymic activity in the wildtype microorganism. This may be achieved by a mutation and/or overexpression of a gene coding for the enzyme having the function of a carbamoyl phosphate synthase.

Furthermore, in the microorganism according to the present invention at least one or more of the genes coding for an enzyme of the biosynthetic pathway of L-omithine and L-arginine, comprising argF/argF2 coding for an ornithine carbamoyl transferase, argG coding for an argininosuccinate synthetase and argH coding for an argininosuccinate lyase may be overexpressed.

Additionally or alternatively, at least one or more of the genes coding for an enzyme of the biosynthetic pathway of L-omithine and L-arginine, comprising gdh coding for a glutamate dehydrogenase, argJ coding for an ornithine acetyltransferase, argB coding for an acetyl glutamate kinase, argC coding for an acetylglutamylphosphate reductase and argD coding for an acetylomithine aminotransferase, may be overexpressed in the microorganism according to the present invention.

Overexpression of a gene is generally achieved by increasing the copy number of the gene and/or by functionally linking the gene with a strong promoter and/or by enhancing the ribosomal binding site and/or by codon usage optimization of the start codon or of the whole gene or a combination comprising a selection of all methods mentioned above.

In the microorganism according to the present invention the gene coding for the protein having the function of an L-arginine:glycine amidinotransferase may be heterologous.

The microorganism according to the present invention is preferably recombinant and the gene coding for the protein having the function of an L-arginine:glycine amidinotransferase (AGAT) is preferably heterologous.

A heterologous gene means that the gene has been inserted into a host organism which does not naturally have this gene. Insertion of the heterologous gene in the host is performed by recombinant DNA technology. Microorganisms that have undergone recombinant DNA technology are called transgenic, genetically modified or recombinant.

In the microorganism of the present invention the gene coding for a protein having the function of an L-arginine:glycine amidinotransferase may further be overexpressed. Overexpression of a gene is generally achieved by increasing the copy number of the gene and/or by functionally linking the gene with a strong promoter and/or by enhancing the ribosomal binding site and/or by codon usage optimization of the start codon or of the whole gene or a combination comprising a selection or all methods mentioned above.

The protein having the function of an L-arginine:glycine amidinotransferase (AGAT) encoded by at least one respective gene in the microorganism of the present invention may e.g. comprise an amino acid sequence which is at least 70% identical, preferably 80% or at least 90% identical to the amino acid sequence according to SEQ ID NO. 13, i.e. the AGAT of Moorea producens 30 (“AGAT_Mpr). In a further embodiment of the present invention the amino acid sequence of the L-arginine:glycine amidinotransferase is identical to amino acid sequence according to SEQ ID NO. 13 (cf. Database UniProt, 15 Feb. 2017, “Glycine amidinotransferase”, XP055706853, EBI accession no. UNIPROT: A0A1D8TKD3). The sequence of wildtype DNA coding for the Moorea producens AGAT is SEQ ID NO: 12, the corresponding DNA sequence that has been codon optimized for C. glutamicum is SEQ ID NO: 14.

The protein having the function of an L-arginine:glycine amidinotransferase encoded by at least one respective gene in the microorganism of the present invention may e.g. also comprise an amino acid sequence which is at least 70% homologous, preferably at least 80% or at least 90% identical to the amino acid sequence of the AGAT of Cylindrospermopsis raciborskii ATW205 (J Muenchhoff et al., FEBS Journal 277 (2010) 3844-3860).

The protein having the function of an L-arginine-glycine amidinotransferase encoded by at least one respective gene in the microorganism of the present invention may comprise an amino acid sequence which is at least 70% homologous, preferably at least 80% or at least 90% identical to the amino acid sequence of the AGAT of Galeopterus variegatus.

The protein having the function of an L-arginine:glycine amidinotransferase in the microorganism of the present invention may comprise an amino acid sequence which is at least 70% homologous, preferably at least 80% or at least 90% homologous to the amino acid sequence of the AGAT of Homo sapiens, e.g. the AGAT of Homo sapiens itself (A. Humm, Biochem. J. (1997) 322, 771-776) or the AGAT of Rattus norvegicus.

Generally, the overexpression of a gene, according to the present invention, is achieved by increasing the copy number of the gene and/or by an enhancement of regulatory factors, e.g. by functionally linking the gene with a strong promoter and/or by enhancing the ribosomal binding site and/or by codon usage optimization of the start codon or of the whole gene. The enhancement of such regulatory factors which positively influence gene expression can, for example, be achieved by modifying the promoter sequence upstream of the structural gene in order to increase the effectiveness of the promoter or by completely replacing said promoter with a more effective or a so-called strong promoter. Promoters are located upstream of the gene. A promoter is a DNA sequence consisting of about 40 to 50 base pairs and which constitutes the binding site for an RNA polymerase holoenzyme and the transcriptional start point, whereby the strength of expression of the controlled polynucleotide or gene can be influenced. Generally, it is possible to achieve an overexpression or an increase in the expression of genes in bacteria by selecting strong promoters, for example by replacing the original promoter with strong, native (originally assigned to other genes) promoters or by modifying certain regions of a given, native promoter (for example its so-called −10 and −35 regions) towards a consensus sequence, e.g. as taught by M. Patek et al. (Microbial Biotechnology 6 (2013), 103-117) for C. glutamicum. An example for a “strong” promoter is the superoxide dismutase (sod) promoter (“Psod”; Z. Wang et al., Eng. Life Sci. 2015, 15, 73-82). A “functional linkage” is understood to mean the sequential arrangement of a promoter with a gene, which leads to a transcription of the gene.

The genetic code is degenerated which means that a certain amino acid may be encoded by a number of different triplets. The term codon usage refers to the observation that a certain organism will typically not use every possible codon for a certain amino acid with the same frequency Instead, an organism will typically show certain preferences for specific codons meaning that these codons are found more frequently in the coding sequence of transcribed genes of an organism. If a certain gene foreign to its future host, i.e. from a different species, should be expressed in the future host organism the coding sequence of said gene should then be adjusted to the codon usage of said future host organism (i.e. codon usage optimization).

Table 1 shows the different names of enzymes involved in or contributing to arginine biosynthesis in different species, i.e. E. coli. C. glutamicum and Pseudomonas putida (P. putida)

TABLE 1
Names of Enzymes in different species

| Enzyme name | Alias | EC Number | E. coli | C. glutamicum | P. putida |
|---|---|---|---|---|---|
| glutamate | NADP-SPECIFIC GLUTAMATE | 1.4.1.4 | gdhA | gdh | gdhA/ |
| | | | | | gdhB |
| dehydrogenase | DEHYDROGENASE | (1.4.1.2 in | | | |
| | | P. putida) | | | |
| N-acetyl glutamokinase | ACETYLGLUTAMATE KINASE | 2.7.2.8 | argB | argB | argB |
| N-acetylglutamyl | N-acetyl-gamma- | 1.2.1.38 | argC | argC | argC |
| phosphate reductase | glutamylphosphate | | | | |
| | reductase | | | | |
| N-acetylglutamic acid- | ACETYLORNITHINE | 2.6.1.11/ | argD | argD | aruC |
| γ-semialdehyde | AMINOTRANSFERASE | 2.6.1.17 | | | |
| dehydrogenase | (in e.c. | | | | |
| | bifunctional | | | | |
| | acetylornithine | | | | |
| | aminotransferase/ | | | | |
| | succinyldiaminopimelate | | | | |
| | aminotransferase) | | | | |
| acetylornithine | bifunctional | 2.3.1.35/ | | argJ | argJ |
| deacetylase | acetylornithine | 2.3.1.1 | | | |
| | deacetylase/GLUTAMATE | | | | |
| | N-ACETYLTRANSFERASE | | | | |
| acetylornithine | | 3.5.1.16 | argE | | argE |
| deacetylase | | | | | |
| | GLUTAMATE N- | 2.3.1.1 | argA | | argA |
| | ACETYLTRANSFERASE | | | | |
| ornithine | ornithine | 2.1.3.3 | argI | argF/ | arcB/ |
| carbamoyltransferase | carbamoyltransferase 1 | | | argF2 | argF |
| Argininosuccinate | ARGININOSUCCINATE | 6.3.4.5 | argG | argG | argG |
| synthetase | SYNTHASE | | | | |
| Argininosuccinate | ARGININOSUCCINATE | 4.3.2.1 | argH | argH | argH |
| lyase | LYASE | | | | |
| carbamoyl-phosphate | carbamoyl-phosphate | 6.3.5.5 | carAB | carAB | carAB |
| synthase | synthase | | | | |
| carbamate kinase | carbamate kinase | 2.7.2.2 | ybcF/ | | arcC |
| | | | yqeA | | |

In the microorganism of the present invention the gene coding for the protein having the function of an arginine exporter may be inactivated or deleted. Furthermore, in the microorganism of the present invention, a gene coding for a transcriptional activator of the gene coding for the protein having the function of an arginine exporter may be deleted.

The microorganism of the present invention may belong to the genus Corynebacterium, preferably Corynebacterium glutamicum (C. glutamicum), or to the genus Enterobacteriaceae, preferably Escherichia coli (E. coli), or to the genus Pseudomonas, preferably Pseudomonas putida (P. putida).

In Corynebacterium glutamicum the gene coding for the protein having the function of an arginine exporter is lysE and the gene coding for the transcriptional activator is lysG. In Escherichia coli the gene coding for the protein having the function of an arginine exporter is argO (ybjE). In Pseudomonas putida the protein having the function of an arginine exporter is lysE.

The above-mentioned problem is further solved by a method for the fermentative production of guanidino acetic acid (GAA), comprising the steps of a) cultivating the microorganism according to the present invention as defined above in a suitable medium under suitable conditions, and b) accumulating GAA in the medium to form an GAA containing fermentation broth.

The method according to the present invention may further comprise adding glycine and/or adding L-arginine and/or adding L-omithine to the medium. Preferably, the medium is supplemented with glycine in a concentration ranging from 0.1 to 300 g glycine/l medium, preferably 0.82 g glycine/l medium, and/or with L-arginine to obtain a concentration ranging from 0.1 to 200 g L-arginine/l medium, preferably 1.9 g L-arginine/l medium.

The method of the present invention may further comprise the step of isolating GAA from the fermentation broth.

The method according to the present invention may further comprise the step of drying and/or granulating the GAA containing fermentation broth.

The present invention further concerns a microorganism as defined above, further comprising a gene coding for an enzyme having the activity of a guanidinoacetate N-methyltransferase (EC: 2.1.1.2). Preferably, the gene coding for an enzyme having the activity of a guanidinoacetate N-methyltransferase is overexpressed.

The present invention also concerns a method for the fermentative production of creatine, comprising the steps of a) cultivating the microorganism according to the present invention comprising a gene coding for an enzyme having the activity of a guanidinoacetate N-methyltransferase in a suitable medium under suitable conditions, and b) accumulating creatine in the medium to form a creatine containing fermentation broth.

Preferably, the method further comprises isolating creatine from the creatine containing fermentation broth, creatine may be extracted from fermentation broth by isoelectric point method and/or ion exchange method. Alternatively, creatine can be further purified by a method of recrystallization in water.

### EXPERIMENTAL SECTION

### a) MATERIALS and METHODS

### Chemicals

Kanamycin solution from Streptomyces kanamyceticus was purchased from Sigma Aldrich (St. Louis, USA, Cat. no. K0254). If not stated otherwise, all other chemicals were purchased analytically pure from Merck (Darmstadt, Germany), Sigma Aldrich (St. Louis, USA) or Carl-Roth (Karlsruhe, Germany).

### Cultivation for Cell Proliferation

If not stated otherwise, cultivation/incubation procedures were performed as follows herewith:
a. LB broth (MILLER) from Merck (Darmstadt, Germany; Cat. no. 110285) was used to cultivate E. coli strains in liquid medium. The liquid cultures (10 ml liquid medium per 100 ml Erlenmeyer flask with 3 baffles) were incubated in the Infors HT Multitron standard incubator shaker from Infors GmbH (Bottmingen, Switzerland) at 30° C. and 200 rpm.
b. LB agar (MILLER) from Merck (Darmstadt, Germany, Cat. no. 110283) was used for cultivation of E. coli strains on agar plates. The agar plates were incubated at 30° C. in an INCU-Line® mini incubator from VWR (Radnor, USA).
c. Brain heart infusion broth (BHI) from Merck (Darmstadt, Germany, Cat. no. 110493) was used to cultivate C. glutamicum strains in liquid medium. The liquid cultures (10 ml liquid medium per 100 ml Erdenmeyer flask with 3 baffles) were incubated in the Infors HT Multitron standard incubator shaker from Infors GmbH (Bottmingen, Switzerland) at 30° C. and 200 rpm.
d. Brain heart agar (BHI-agar) from Merck (Darmstadt, Germany, Cat. no. 113825) was used for cultivation of C. glutamicum strains on agar plates. The agar plates were incubated at 30° C. in an incubator from Heraeus Instruments with Kelvitron® temperature controller (Hanau, Germany).
e. For cultivating C. glutamicum after electroporation, BHI-agar (Merck, Darmstadt, Germany, Cat. no. 113825) was supplemented with 134 g/l sorbitol (Carl Roth GmbH+Co. KG, Karlsruhe, Germany), 2.5 g/l yeast extract (Oxoid/ThermoFisher Scientific, Waltham, USA, Cat. no. LP0021) and 25 mg/l kanamycin. The agar plates were incubated at 30° C. in an incubator from Heraeus Instruments with Kelvitron® temperature controller (Hanau, Germany).

### Determining Optical Density of Bacterial Suspensions

a. The optical density of bacterial suspensions in shake flask cultures was determined at 600 nm (OD600) using the Bio-Photometer from Eppendorf AG (Hamburg, Germany).

b. The optical density of bacterial suspensions produced in the Wouter Duetz (WDS) micro fermentation system (24-Well Plates) was determined at 660 nm (OD660) with the GENios™ plate reader from Tecan Group AG (Männedorf, Switzerland).

### Centrifugation

a. Bacterial suspensions with a maximum volume of 2 ml were centrifuged in 1.5 ml or 2 ml reaction tubes (e.g. Eppendorf Tubes® 3810X) using an Eppendorf 5417 R benchtop centrifuge (5 min. at 13.000 rpm).

b. Bacterial suspensions with a maximum volume of 50 ml were centrifuged in 15 ml or 50 ml centrifuge tubes (e.g. Falcon™ 50 ml Conical Centrifuge Tubes) using an Eppendorf 5810 R benchtop centrifuge for 10 min. at 4.000 rpm.

### DNA Isolation

Plasmid DNA was isolated from E. coli cells using the QIAprep Spin Miniprep Kit from Qiagen (Hilden, Germany, Cat. No. 27106) according to the instructions of the manufacturer.

### Polymerase Chain Reaction (PCR)

PCR with a proof reading (high fidelity) polymerase was used to amplify a desired segment of DNA for Sanger sequencing or DNA assembly. Non-proof-reading polymerase Kits were used for determining the presence or absence of a desired DNA fragment directly from E. coli or C. glutamicum colonies. a. The Phusion® High-Fidelity DNA Polymerase Kit (Phusion Kit) from New England BioLabs Inc. (lpswich, USA, Cat. No. M0530) was used for template-correct amplification of selected DNA regions according to the instructions of the manufacturer (see Table 2).

TABLE 2
Thermocycling conditions for PCR with Phusion ® High-Fidelity
DNA Polymerase Kit from New England BioLabs Inc.
PCR Program

| | Time | T | |
|---|---|---|---|
| Step | [min.:sec.] | [° C.] | Description |
| 1 | 00:30 | 98 | Initial denaturation step |
| 2 | 00:05 | 98 | Denaturation step |
| 3 | 00:30 | 60 | Annealing step |
| 4 | 00:xx | 72 | Elongation step |
| | | | 1 min. per kb DNA |
| | | | Repeat step 2 to 4: 35 x |
| 5 | 05:00 | 72 | Final elongation step |
| 6 | Hold | 4 | Cooling step |

b. Taq PCR Core Kit (Taq Kit) from Qiagen (Hilden, Germany. Cat. No. 201203) was used to amplify a desired segment of DNA in order to confirm its presence. The kit was used according to the instructions of the manufacturer (see Table 3).

TABLE 3
Thermocycling conditions for PCR with Taq
PCR Core Kit (Taq Kit) from Qiagen.
PCR Program

| | Time | T | |
|---|---|---|---|
| Step | [min.:sec.] | [° C.] | Description |
| 1 | 05:00 | 94 | Initial denaturation step |
| 2 | 00:30 | 94 | Denaturation step |
| 3 | 00:30 | 52 | Annealing step |
| 4 | 01:20 | 72 | Elongation step |
| | | | 1 min. per kb DNA |
| | | | Repeat step 2 to 4: 35 x |
| 5 | 04:00 | 72 | Final elongation step |
| 6 | Hold | 4 | Cooling step |

c. SapphireAmp® Fast PCR Master Mix (Sapphire Mix) from Takara Bio Inc (Takara Bio Europe S.A.S., Saint-Germain-en-Laye, France, Cat. No. RR350A/B) was used as an alterative to confirm the presence of a desired segment of DNA in cells taken from E. coli or C. glutamicum colonies according to the instructions of the manufacturer (see Table 4).

TABLE 4
Thermocycling conditions for PCR with SapphireAmp ® Fast
PCR Master Mix (Sapphire Mix) from Takara Bio Inc.
PCR Program

| | Time | T | |
|---|---|---|---|
| Step | [min.:sec.] | [° C.] | Description |
| 1 | 01:00 | 94 | Initial denaturation step |
| 2 | 00:05 | 98 | Denaturation step |
| 3 | 00:05 | 55 | Annealing step |
| 4 | 00:05 | 72 | Elongation step |
| | | | Repeat step 2 to 4: 30 x |
| 5 | 04:00 | 72 | Final elongation step |
| 6 | Hold | 4 | Cooling step |

d. All oligonucleotide primers were synthesized by Eurofins Genomics GmbH (Ebersberg, Germany) using the phosphoramidite method described by McBride and Caruthers (1983).

e. As PCR template either a suitably diluted solution of isolated plasmid DNA or of total DNA isolated from a liquid culture or the total DNA contained in a bacterial colony (colony PCR) was used. For said colony PCR the template was prepared by taking cell material with a toothpick from a colony on an agar plate and placing the cell material directly into the PCR reaction tube. The cell material was heated for 10 sec. with 800 W in a microwave oven type Mikrowave & Grill from SEVERIN Elektrogeralte GmbH (Sundem, Germany) and then the PCR reagents were added to the template in the PCR reaction tube.

f. All PCR reactions were carried out in PCR cyclers type Mastercycler or Mastercycler nexus gradient from Eppendorf AG (Hamburg, Germany).

### Restriction Enzyme Digestion of DNA

For restriction enzyme digestions either “FastDigest restriction endonucleases (FD)” (ThermoFisher Scientific, Waltham, USA) or restriction endonucleases from New England BioLabs Inc. (Ipswich, USA) were used. The reactions were carried out according to the instructions of the manufacturer's manual.

### Determining the Sizes of DNA Fragments

a. The sizes of small DNA fragments (<1000 bps) were usually determined by automatic capillary electrophoresis using the QIAxcel from Qiagen (Hilden, Germany).

b. If DNA fragments needed to be isolated or if the DNA fragments were >1000 bps DNA was separated by TAE agarose gel electrophoresis and stained with GelRed® Nucleic Acid Gel Stain (Biotium, Inc., Fremont, Canada). Stained DNA was visualized at 302 nm.

### Purification of PCR Amplificates and Restriction Fragments

PCR amplificates and restriction fragments were cleaned up using the QIAquick PCR Purification Kit from Qiagen (Hilden, Germany; Cat. No. 28106), according to the manufacturer's instructions. DNA was eluted with 30 μl 10 mM Tris*HCl (pH 8.5).

### Determining DNA Concentration

DNA concentration was measured using the NanoDrop Spectrophotometer ND-1000 from PEQLAB BioTechnology GmbH, since 2015 VWR brand (Erlangen, Germany).

### Assembly Cloning

Plasmid vectors were assembled using the “NEBuilder HiFi DNA Assembly Cloning Kit” purchased from New England BioLabs Inc. (Ipswich, USA, Cat. No. E5520). The reaction mix, containing the linear vector and at least one DNA insert, was incubated at 50° C. for 60 min. 0.5 μl of Assembly mixture was used for each transformation experiment.

Chemical Transformation of E. coli

For plasmid cloning, chemically competent “NEB® Stable Competent E. coli (High Efficiency)” (New England BioLabs Inc., Ipswich, USA, Cat. No. C3040) were transformed according to the manufacturers protocol. Successfully transformed cells were selected on LB agar supplemented with 25 mg/l kanamycin.

Transformation of C. glutamicum

Transformation of C. glutamicum with plasmid-DNA was conducted via electroporation using a “Gene Pulser Xcell” (Bio-Rad Laboratories GmbH, Feldkirchen, Germany) as described by Ruan et al. (2015). Electroporation was performed in 1 mm electroporation cuvettes (Bio-Rad Laboratories GmbH, Feldkirchen, Germany) at 1.8 kV and a fixed time constant set to 5 ms. Transformed cells were selected on BHI-agar containing 134 g/l sorbitol, 2.5 g/l Yeast Extract and 25 mg/l kanamycin.

C. glutamicum Strains

Corynebacterium glutamicum ATCC13032 (DSM 20300, Kinoshita S, Udaka S, Shimono M., J. Gen. Appl. Microbiol. 1957; 3(3): 193-205), the C. glutamicum wild type strain, is commercially available at the American Type Culture Collection (ATCC) or at the DSMZ-German Collection of Microorganisms and Cell Cultures GmbH.

Corynebacterium glutamicum ATCC21831, an L-arginine producing C. glutamicum strain (U.S. Pat. No. 3,849,250 A), is commercially available at the American Type Culture Collection (ATCC).

### Determining Nucleotide Sequences

Nucleotide sequences of DNA molecules were determined by Eurofins Genomics GmbH (Ebersberg, Germany) by cycle sequencing, using the dideoxy chain termination method of Sanger et al. (Proceedings of the National Academy of Sciences USA 74, 5463-5467, 1977). Clonemanager Professional 9 software from Scientific & Educational Software (Denver, USA) was used to visualise and evaluate the sequences.

Glycerol Stocks of E. coli and C. glutamicum Strains

For long time storage of E. coli- and C. glutamicum strains glycerol stocks were prepared. Selected E. coli clones were cultivated in 10 ml LB medium supplemented with 2 g/l glucose. Selected C. glutamicum clones were cultivated in 10 ml twofold concentrated BHI medium supplemented with 2 g/l glucose. Media for growing plasmid containing E. coli- and C. glutamicum strains were supplemented with 25 mg/I kanamycin. The medium was contained in 100 ml Erdenmeyer flasks with 3 baffles. It was inoculated with a loop of cells taken from a colony. The culture was then incubated for 18 h at 30° C. and 200 rpm. After said incubation period 1.2 ml 85% (v/v) sterile glycerol were added to the culture. The obtained glycerol containing cell suspension was then aliquoted in 2 ml portions and stored at −80° C.

### Gaa Production in Shake Flask Cultivations

Shake flask cultivation in 250 mL baffled Erlenmeyer flasks was used to assess the GAA-production of the strains.

Precultures of the strains were done in 10 ml seed medium (SM). The medium was contained in a 100 ml Erlenmeyer flask. It was inoculated with 100 μl of a glycerol stock culture and the culture was incubated for 24 h at 30° C. and 200 rpm. The composition of the seed medium (SM) is shown in Table 5. Kanamycin was added to cultures were needed to retain plasmids.

TABLE 5
Seed medium (SM)

| Components | Concentration (g/l) |
|---|---|
| (NH₄)₂SO₄ | 20 |
| Yeast extract FM902 (Angel Yeast Co., LTD, | 20 |
| Hubei, P.R.China) | |
| KH₂PO₄ | 1 |
| Mg-Sulfat * 7 H₂O | 0.5 |
| Glucose | 20 |
| Kanamycin | 0.025 |
| pH = 7.0 | |

After said incubation period the optical densities OD600 of the precultures were determined. The volume, needed to inoculate 2.5 ml of production medium (PM) to an OD600 of 0.5, was sampled from the preculture, centrifuged (1 min at 8000 g) and the supernatant was discarded. Cells were then resuspended in 200 μl of production medium.

The main cultures were started by inoculating the 2.4 ml production medium (PM) containing wells of the 24 Well WDS-Plate with each 100 μl of the resuspended cells from the precultures. The composition of the production medium (PM) is shown in Table 6.

TABLE 6
Production medium (PM)

| Components | Concentration (g/l) |
|---|---|
| Yeast extract FM902 (Angel Yeast Co., LTD, | 8 |
| Hubei, P.R.China) | |
| (NH₄)₂SO₄ | 40 |
| KH₂PO₄ | 1.5 |
| MgSO₄ * 7 H₂O | 0.5 |
| FeSO₄ * 7 H₂O | 0.02 |
| MnSO₄ * H₂O | 0.02 |
| Calcium carbonate | 20 |
| Glucose | 100 |
| Kanamycin | 0.025 |
| L-arginine | 5 mM |
| Glycine | 20 |
| pH = 7.2 | |

The main cultures were incubated for 48 h at 30° C. and 200 rpm in an Infors HT Multitron standard incubator shaker from Infors GmbH (Bottmingen, Switzerland) until complete consumption of glucose. The glucose concentration in the suspension was analyzed with the blood glucose-meter OneTouch Vita® from LifeScan (Johnson & Johnson Medical GmbH, Neuss, Germany).

After cultivation the culture suspensions were transferred to 50 ml centrifuge tubes (e.g. Falcon™ 50 ml Conical Centrifuge Tubes). A part of the culture suspension was suitably diluted to measure the OD660. Another part of the culture was centrifuged and the concentration of GAA in the supernatant was analyzed as described below.

### Quantification of GAA

Samples were analyzed with an analyzing system from Agilent, consisting of a HPLC “Infinity 1260” coupled with a mass analyzer “Triple Quad 6420” (Agilent Technologies Inc., Santa Clara, USA). Chromatographic separation was done on the Atlantis HILIC Silica column, 4,6λ250 mm, 5 μm (Waters Corporation, Milford, USA) at 35° C. Mobile phase A was water with 10 mM ammonium formate and 0,2% formic acid. Mobile phase B was a mixture of 90% acetonitrile and 10% water, 10 mM ammonium formate were added to the mixture. The HPLC system was started with 100% B, followed by a linear gradient for 22 min and a constant flow rate of 0.6 mL/min to 66% B. The mass analyzer was operated in the ESI positive ionization mode. For detection of GAA the m/z values were monitored by using an MRM fragmentation [M+H]+ 118−76. The limit of quantification (LOQ) for GAA was fixed to 7 ppm.

### B) Experimental Results

### Example 1: Cloning of the Plasmid pK19mobsacB-ΔlysEG for the Chromosomal Deletion of the Genes lysE and lysG in C. Glutamicum Based Strains

The gene lysE codes for an exporter protein that catalyzes the efflux of L-Lysine, L-arginine and L-citrulline in Corynebacterium glutamicum. The expression of lysE is positively regulated by the gene product of lysG. Both genes are located next to each other but are transcribed divergently.

In order to inactivate the transporter protein LysE and the positive regulator protein LysG, the plasmid pK19mobsacB-ΔlysEG (SEQ ID NO: 1) was constructed as described for pK18mobsacB-ΔlysEG in Vrljic et. al 1996 (Vrljic, M., et al. (1996). “A new type of transporter with a new type of cellular function: L-lysine export from Corynebacterium glutamicum.” Mol Microbiol 22(5): 815-826; https://doi.org/10.1046/j.1365-2958.1996.01527.x).

### Example 2: Chromosomal Deletion of the Gene argR in ATCC13032

To improve intracellular L-Arginine formation and L-Arginine recycling from L-Ornithine, the gene argR coding for the central repressor protein ArgR controlling the L-arginine biosynthetic pathway was inactivated.

Therefore, the plasmid pKl8mobsacB_DargR was constructed as follows. Plasmid pK18mobsacB (Schäfer, 1994) was cut using Xbal and the linearized vector DNA (5721 bps) was purified using the “QIAquick Gel Extraction Kit” (Qiagen GmbH, Hilden, Germany).

For constructing the insert, two DNA fragments were created by PCR with the following pairs of primers (genomic DNA of ATCC13032 as template):
DargR_If (SEQ ID NO: 2), +DargR_Ir (SEQ ID NO: 3)=left homology arm (983 bps)
DargR_rf (SEQ ID NO: 4), +DargR_jr (SEQ ID NO: 5)=left homology arm (984 bps)

The product DNAs were purified using the “QIAquick PCR Purification Kit” (Qiagen GmbH, Hilden, Germany).

The linearized plasmid and the PCR products were then assembled using the “NEBuilder HiFi DNA Assembly Cloning Kit” (New England BioLabs Inc., Ipswich, USA, Cat. No. E5520). The resulting deletion vector was named pK18mobsacB_DargR. It was verified by restriction enzyme digestion and DNA sequencing.

For deleting the argR gene, pK18mobsacB_DargR was transformed into ATCC13032 by electroporation. Chromosomal integration (resulting from a first recombination event) was selected by plating on BHI agar supplemented with 134 g/l sorbitol, 2.5 g/l yeast extract and 25 mg/l kanamycin. The agar plates were incubated for 48 h at 33° C.

Individual colonies were transferred onto fresh agar plates (with 25 mg/l kanamyan) and incubated for 24 h at 33° C. Liquid cultures of these clones were cultivated for 24 h at 33° C. in 10 ml BHI medium contained in 100 ml Erlenmeyer flasks with 3 baffles. To isolate clones that have encountered a second recombination event, an aliquot was taken from each liquid culture, suitably diluted and plated (typically 100 to 200 μl) on BHI agar supplemented with 10% saccharose. These agar plates were incubated for 48 h at 33° C. Colonies growing on the saccharose containing agar plates were then examined for kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined by PCR and DNA sequencing. The resulting strain was named ATCC13032_DargR.

### Example 3: Chromosomal Insertion of the Sod Promoter Upstream of the carAB Operon in ATCC13032_DargR

To improve the production of L-arginine, the strong sod-promoter was inserted into the genome of ATCC13032_DargR upstream of the carAB operon. Therefore, the plasmid pK18mobsacB_Psod-carAB was constructed as follows. pK18mobsacB was cut using EcoRI+HindIII and the linearized vector DNA (5670 bps) was cut out of an agarose gel. The DNA was extracted using the “QIAquick PCR Purification Kit” (Qiagen GmbH, Hilden, Germany).

For constructing the insert, three DNA fragments were created by PCR with the following pairs of primers (genomic DNA of ATCC13032 as template):
PsodcarAB-LA-F (SEQ ID NO: 6)+PsodcarAB-LA-R (SEQ ID NO: 7)=left homology arm (1025 bps)
PsodcarAB-F (SEQ ID NO: 8)+PsodcarAB-R (SEQ ID NO: 9)=sod-promoter (250 bps)
PsodcarAB-RA-F (SEQ ID NO: 10)+PsodcarAB-RA-R (SEQ ID NO: 11)=right homology arm (944 bps)

The product DNAs were purified using the “QIAquick PCR Purification Kit” (Qiagen GmbH, Hidlen, Germany). The linearized plasmid and the PCR products were then assembled using the “NEBuilder HiFi DNA Assembly Cloning Kit” (New England BioLabs Inc., Ipswich, USA, Cat. No. E5520). Proper plasmid clones were identified by restriction digestion and DNA sequencing.

The resulting plasmid pK18mobsacB_Psod-carAB was then transformed into ATCC13032_DargR by electroporation. Chromosomal integration (resulting from a first recombination event) was selected by plating on BHI agar supplemented with 134 g/l sorbitol, 2.5 g/l yeast extract and 25 mg/l kanamycin. The agar plates were incubated for 48 h at 33° C.

Individual colonies were transferred onto fresh agar plates (with 25 mg/l kanamycin) and incubated for 24 h at 33° C. Liquid cultures of these clones were cultivated for 24 h at 33° C. in 10 ml BHI medium contained in 100 ml Erlenmeyer flasks with 3 baffles. To isolate clones that have encountered a second recombination event, an aliquot was taken from each liquid culture, suitably diluted and plated (typically 100 to 200 μl) on BHI agar supplemented with 10% saccharose. These agar plates were incubated for 48 h at 33° C. The colonies growing on the saccharose containing agar plates were then examined for kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined by PCR and DNA sequencing for the appropriate integration of the sod promoter. The resulting strain was named ATCC13032_DargR_Psod-carAB.

### Example 4: Chromosomal Deletion of the Genes lysE and lysG in ATCC13032_DargR_Psod-carAB

The gene lysE codes for an exporter protein that catalyzes the efflux of L-Lysine, L-arginine and L-citrulline in Corynebactedum glutamicum. The expression of lysE is positively regulated by the gene product of lysG. Both genes are located next to each other but are transcribed divergently. For deleting the genes lysE and lysG gene, pK19mobsacB_DlysEG (see example 1) was transformed into ATCC13032_DargR_Psod-carAB by electroporation. Chromosomal integration (resulting from a first recombination event) was selected by plating on BHI agar supplemented with 134 g/l sorbitol, 2.5 g/l yeast extract and 25 mg/l kanamycin. The agar plates were incubated for 48 h at 33° C.

Individual colonies were transferred onto fresh agar plates (with 25 mg/l kanamycin) and incubated for 24 h at 33° C. Liquid cultures of these clones were cultivated for 24 h at 33° C. in 10 ml BHI medium contained in 100 ml Erlenmeyer flasks with 3 baffles. To isolate clones that have encountered a second recombination event, an aliquot was taken from each liquid culture, suitably diluted and plated (typically 100 to 200 μl) on BHI agar supplemented with 10% saccharose. These agar plates were incubated for 48 h at 33° C. Colonies growing on the saccharose containing agar plates were then examined for kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined by PCR and DNA sequencing. The resulting strain was named ATCC13032_DargR_Psod-carAB_DlysEG.

### Example 5: Cloning of the Gene AGAT-Mp Coding for an L-Arginine:Glycine Amidinotransferase (AGAT, EC 2.1.4.1) from Moorea producens

Moorea producens is a filamentous cyanobacterium. The genome of the Moorea producens strain PAL-8-15-08-1 was published by Leao et al. (Leao T, Castelgo G, Korobeynikov A. Monroe E A, Podell S, Glukhov E, Allen E E, Gerwick W H, Gerwick L. Proc Natl Acad Sci USA. 2017 Mar. 21; 114(12):3198-3203. doi: 10.1073/pnas.1618556114; Genbank accession Number CP017599.1). It contains an open reading frame coding for a L-arginine:glycine amidinotransferase (AGAT, EC 2.1.4.1; locus_tag BJP34_00300 shown in SEQ ID NO: 12). SEQ ID NO: 13 shows the derived amino acid sequence (Genbank accession Number WP_070390602).

Using the software tool “Optimizer” (http://genomes.urv.es/OPTIMIZER/) this amino acid sequence was translated back into a DNA sequence optimized for the codon usage of C. glutamicum (SEQ ID NO: 14).

A segment of the optimized gene, consisting of base pairs 13-1142, was expanded with a BsmBI restriction site at its 5′-end. At the 3′-end a second stop-codon, the lysS-terminator from C. glutamicum and a BsmBI restriction site were added. The resulting DNA sequence (SEQ ID NO: 15) was ordered for gene synthesis from Invitrogen/Geneart (Thermo Fisher Scientific, Waltham, USA) and it was delivered as part of a cloning plasmid with an ampicillin resistance gene (designated as pMA-RQ_AGAT_Mp_opt). A second DNA segment was designed, consisting of a sequence for assembly cloning, a promoter sequence, a ribosomal binding site and the first 81 nucleotides of the optimized AGAT-Mp gene. The sequence was ordered as a linear DNA string for gene synthesis from Invitrogen/Geneart (Thermo Fisher Scientific, Waltham, USA; SEQ ID NO: 16).

### Example 6: Cloning of the Expression Plasmid pLIB_pBL1_AGAT-Mp

The E. coli-C. glutamicum shuttle plasmid pLIB_pBL1 has the replication origin from pBL1, the pSC101 replication origin, a kanamycin resistance gene and the BioBricks Terminator BBa_B1006 downstream of a Notl restriction site (SEQ ID NO: 17). It was digested using the restriction endonuclease Notl and the DNA was purified with the “QIAquick PCR Purification Kit” (Qiagen GmbH, Hilden, Germany).

Using the primers AGAT_f (SEQ ID NO: 18) and AGAT_r (SEQ ID NO: 19) a DNA fragment was amplified by PCR with pMA-RQ_AGAT_Mp_opt as a template. The PCR-product was purified using the “QIAquick PCR Purification Kit” (Qiagen GmbH, Hilden, Germany).

The linearized plasmid, the promoter containing DNA-string and the PCR product were assembled using the “NEBuilder HiFi DNA Assembly Cloning Kit” (New England BioLabs Inc., Ipswich, USA, Cat. No. E5520). The assembly product was transformed into “NEB Stable Competent E. coli (High Efficiency)” (New England Biolabs, Ipswich, USA) and cells were grown on LB agar containing 25 mg/l kanamycin. Proper plasmid clones were identified by restriction digestion and DNA sequencing. The resulting plasmid was named pLIB_pBL1_AGAT-Mp.

### Example 7: Chromosomal Expression of the Arginine Biosynthesis Genes argF, argG, argH Under The Control of the Strong Constitutive Promoter Pg3

For enhancing the activities of ArgF, ArgG and ArgH, additional copies of the corresponding genes were inserted into the genome. A synthetic operon was designed, consisting of Pg3, argF, argG, argH and flanking regions for genomic integration (SEQ ID NO: 20).

The DNA sequence was ordered for gene synthesis from Invitrogen/Geneart (Thermo Fisher Scientific, Waltham. USA) and it was delivered as part of a cloning plasmid with an ampicillin resistance gene (designated as pMA-RQ_argFGH).

Using the primers argFGH_f (SEQ ID NO: 21) and argFGH_r (SEQ ID NO: 22) a DNA fragment was amplified by PCR with pMA-RQ_argFGH as a template. The PCR-product was purified using the “QIAquick PCR Purification Kit” (Qiagen GmbH, Hilden, Germany).

The plasmid pK18mobsacB (Schäfer, A. et al., Gene. 1994 Jul. 22; 145(1):69-73. doi: 10.1016/0378-1119(94)90324-7) was cut using EcoRI+HindIII and the linearized vector DNA (5870 bps) was cut out of an agarose gel. The DNA was extracted using the “QIAquick PCR Purification Kit” (Qiagen GmbH, Hilden, Germany).

The linearized plasmid and the PCR-product were assembled using the “NEBuilder HiFi DNA Assembly Cloning Kit” (New England BioLabs Inc., Ipswich, USA, Cat. No. E5520). The assembly product was transformed into “NEB Stable Competent E. coli (High Efficiency)” (New England Biolabs, Ipswich, USA) and cells were grown on LB agar containing 25 mg/l kanamycin. Proper plasmid clones were identified by restriction digestion and DNA sequencing. The resulting plasmid was named pK18_IBcg0054::Pg3-argFGH (SEQ ID NO: 23).

For inserting the synthetic operon, pK18_IBcg0054::Pg3-argFGH was transformed into ATCC13032_DargR by electroporation. Chromosomal integration (resulting from a first recombination event) was selected by plating on BHI agar supplemented with 134 g/l sorbitol, 2.5 g/l yeast extract and 25 mg/l kanamycin. The agar plates were incubated for 48 h at 33° C.

Individual colonies were transferred onto fresh agar plates (with 25 mg/l kanamycin) and incubated for 24 h at 33° C. Liquid cultures of these clones were cultivated for 24 h at 33° C. in 10 ml BHI medium contained in 100 ml Erlenmeyer flasks with 3 baffles. To isolate clones that have encountered a second recombination event, an aliquot was taken from each liquid culture, suitably diluted and plated (typically 100 to 200 μl) on BHI agar supplemented with 10% saccharose. These agar plates were incubated for 48 h at 33° C. Colonies growing on the saccharose containing agar plates were then examined for kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined by PCR and DNA sequencing. The resulting strain was named ATCC13032_DargR_IBcg0054::Pg3-argFGH.

### Example 8: Chromosomal Deletion of the Genes lysE and lysG in Atcc13032_DargR_Bcg0054::Pg3-argFGH

The gene lysE codes for an exporter protein that catalyzes the efflux of L-Lysine, L-arginine and L-citrulline in Corynebacterium glutamicum. The expression of lysE is positively regulated by the gene product of lysG. Both genes are located next to each other but are transcribed divergently.

For deleting the genes lysE and lysG gene, pK19mobsacB_DlysEG (see example 1) was transformed into ATCC13032_DargR_IBcg0054::Pg3-argFGH by electroporation. Chromosomal integration (resulting from a first recombination event) was selected by plating on BHI agar supplemented with 134 g/l sorbitol, 2.5 g/l yeast extract and 25 mg/l kanamycin. The agar plates were incubated for 48 h at 33° C.

Individual colonies were transferred onto fresh agar plates (with 25 mg/l kanamycin) and incubated for 24 h at 33° C. Liquid cultures of these clones were cultivated for 24 h at 33° C. in 10 ml BHI medium contained in 100 ml Erlenmeyer flasks with 3 baffles. To isolate clones that have encountered a second recombination event, an aliquot was taken from each liquid culture, suitably diluted and plated (typically 100 to 200 μl) on BHI agar supplemented with 10% saccharose. These agar plates were incubated for 48 h at 33° C. Colonies growing on the saccharose containing agar plates were then examined for kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined by PCR and DNA sequencing. The resulting strain was named ATCC13032_DargR_IBcg0054::Pg3-argFGH_DlysEG.

### Example 9: Chromosomal Deletion of the Genes lysE and lysG in ATCC21831_DlysEG

The gene lysE codes for an exporter protein that catalyzes the efflux of L-Lysine, L-arginine and L-citrulline in Corynebacterium glutamicum. The expression of lysE is positively regulated by the gene product of lysG. Both genes are located next to each other but are transcribed divergently.

For deleting the genes lysE and lysG gene, pK19mobsacB_DlysEG (see example 1) was transformed into ATCC21831_DlysEG by electroporation. Chromosomal integration (resulting from a first recombination event) was selected by plating on BHI agar supplemented with 134 g/l sorbitol, 2.5 g/l yeast extract and 25 mg/l kanamycin. The agar plates were incubated for 48 h at 33° C.

Individual colonies were transferred onto fresh agar plates (with 25 mg/l kanamycin) and incubated for 24 h at 33° C. Liquid cultures of these clones were cultivated for 24 h at 33° C. in 10 ml BHI medium contained in 100 ml Erlenmeyer flasks with 3 baffles. To isolate clones that have encountered a second recombination event, an aliquot was taken from each liquid culture, suitably diluted and plated (typically 100 to 200 μl) on BHI agar supplemented with 10% saccharose. These agar plates were incubated for 48 h at 33° C. Colonies growing on the saccharose containing agar plates were then examined for kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined by PCR. The resulting strain was named ATCC2183_DlysEG.

### Example 10: Transformation of C. glutamicum Strains with pLIB_pBL1_AGAT-MP

The following strains of C. glutamicum were transformed with pLIB_pBL1_AGAT-Mp by electroporation and plasmid containing cells were selected with 25 mg/l kanamycin. The resulting plasmid containing strains are shown in Table 7.

TABLE 7
List of plasmid-containing C. glutamicum strains

| Plasmid | Recipient strain | Resulting strain |
|---|---|---|
| pLIB_pBL1 | ATCC13032 | ATCC13032/pLIB_pBL1 |
| pLIB_pBL1_AGAT-Mp | ATCC13032 | ATCC13032/pLIB_pBL1_AGAT-Mp |
| pLIB_pBL1_AGAT-Mp | ATCC13032_DargR_Psod- | ATCC13032_DargR_Psod-carAB/ |
| | carAB | pLIB_pBL1_AGAT-Mp |
| pLIB_pBL1_AGAT-Mp | ATCC13032_DargR_Psod- | ATCC13032_DargR_Psod- |
| | carAB_DlysEG | carAB_DlysEG/pLIB_pBL1_AGAT- |
| | | Mp |
| pLIB_pBL1_AGAT-Mp | ATCC13032_DargR_IBcg0054::Pg3- | ATCC13032_DargR_IBcg0054::Pg3- |
| | argFGH | argFGH/pLIB_pBL1_AGAT-Mp |
| pLIB_pBL1_AGAT-Mp | ATCC13032_DargR_IBcg0054::Pg3- | ATCC13032_DargR_IBcg0054::Pg3- |
| | argFGH_DlysEG | argFGH_DlysEG/pLIB_pBL1_AGAT- |
| | | Mp |
| pLIB_pBL1 | ATCC21831 | ATCC21831/pLIB_pBL1 |
| pLIB_pBL1_AGAT-Mp | ATCC21831 | ATCC21831/pLIB_pBL1_AGAT-Mp |
| pLIB_pBL1_AGAT-Mp | ATCC21831_DlysEG | ATCC21831_DlysEG/ |
| | | pLIB_pBL1_AGAT-Mp |

C. glutamicum ATCC13032: commonly used wild type strain (Kinoshita et al., J. Gen. Appl. Microbiol. 1957; 3(3): 193-205)
ATCC13032_DargR_Psod-carAB: Increased ability to produce L-arginine due to the reduced activity of the ArgR regulator protein and integration of the strong sod promotor upstream of carAB in ATCC13032.
ATCC13032_DargR_Psod-carAB_DlysEG: Increased ability to produce L-arginine due to the reduced activity of the ArgR regulator protein and integration of the strong sod promotor upstream of carAB in ATCC13032. Reduced export activity of L-arginine.
ATCC13032_DargR_IBcg0054::Pg3-argFGH: Increased ability to produce L-arginine due to the reduced activity of the ArgR regulator protein and genomic integration of an argFGH expression cassette under the control of the strong promoter Pg3 in ATCC13032.
ATCC13032_DargR_IBcg0054::Pg3-argFGH_DlysEG: Increased ability to produce L-arginine due to the reduced activity of the ArgR regulator protein and genomic integration of an argFGH expression cassette under the control of the strong promoter Pg3 in ATCC13032. Reduced export activity of L-arginine.
C. glutamicum strain ATCC21831 (Park et al., Nat Commun. 2014 Aug. 5; 5: 4618) synthesizes L-arginine from primary substrates like ammonia and glucoseC. glutamicum strain ATCC21831_DlysEG (Park et al., Nat Commun. 2014 Aug. 5; 5: 4618) synthesizes L-arginine from primary substrates like ammonia and glucose. Reduced export activity of L-arginine

### Example 11: Impact of Increased Ability to Produce L-Arginine and Reduced Arginine Export on GAA Production

To assess the combined impact of the increased ability to produce L-arginine and reduced L-arginine export, on GAA production, strains ATCC13032, ATCC13032/pLIB_pBL1, ATCC13032/pLIB_pBL1_AGAT-Mp, ATCC13032_DargR_Psod-carAB/pLIB_pBL1_AGAT-Mp, ATCC13032_DargR_Psod-carAB_DlysEG/pLIB_pBL1_AGAT-Mp. ATCC13032_DargR_IBcg0054::Pg3-argFGH/pLIB_pBL1_AGAT-Mp and ATCC13032_DargR_IBcg0054::Pg3-argFGH_DlysEG/pLIB_pBL1_AGAT-Mp were cultivated in the Wouter Duetz system in production medium, and the resulting GAA titers were determined.

TABLE 8
Combined impact of increased ability to produce L-arginine
and reduced Arginine export on GAA production.

| | GAA |
|---|---|
| Strain | (g/L) |
| ATCC13032 | 0 |
| ATCC13032/pLIB_pBL1 | 0 |
| ATCC13032/pLIB_pBL1_AGAT-Mp | 1.1 |
| ATCC13032_DargR_Psod-carAB/pLIB_pBL1_AGAT-Mp | 2.2 |
| ATCC13032_DargR_Psod-carAB_DlysEG/ | 2.3 |
| pLIB_pBL1_AGAT-Mp | |
| ATCC13032_DargR_IBcg0054::Pg3-argFGH/ | 1.7 |
| pLIB_pBL1_AGAT-Mp | |
| ATCC13032_DargR_IBcg0054::Pg3-argFGH_DlysEG/ | 1.9 |
| pLIB_pBL1_AGAT-Mp | |

As shown in Table 8, ATCC13032_DargR_Psod-carAB/pLIB_pBL1_AGAT-Mp and ATCC13032_DargR_IBcg0054::Pg3-argFGH/pLIB_pBL1_AGAT-Mp having a polynucleotide coding for the AGAT from Moorea producens, a deleted argR gene and increased expression of carAB or argFGH genes produced 2.2 g/l or 1.7 g/l of GAA respectively.

Strains ATCC13032_DargR_Psod-carAB_DlysEG/pLIB_pBL1_AGAT-Mp and ATCC13032_DargR_IBcg0054::Pg3-argFGH_DlysEG/pLB_pBL1_AGAT-Mp also have a polynucleotide coding for the AGAT from Moorea producens, a deleted argR gene and increased expression of carAB or argFGH genes. In addition, the genes lysEG are inactivated and they have reduced L-arginine export activity. These strains produced 2.3 g/l and 1.9 g/l GAA respectively, which is improved as compared to strains without reduced L-arginine export.

We conclude that the combination of enzymatic AGAT activity, increased ability to provide L-arginine and reducing L-arginine export improves GAA production.

### Example 12: Impact of Increased Ability to Produce L-Arginine and Reduced Arginine Export on GAA Production Using the L-Arginine Producer Strain ATCC21831

To assess the combined impact of the increased ability to produce L-arginine and reduced L-arginine export, on GAA production, strains ATCC21831, ATCC21831/pLIB_pBL1 and ATCC21831/pLIB_pBL1_AGAT-Mp were cultivated in the Wouter Duetz system in production medium, and the resulting GAA titers were determined.

TABLE 9
Combined impact of increased ability to produce L-arginine
and reduced Arginine export on GAA production using
the L-Arginine producer strain ATCC21831.

| Strain | g/L |
|---|---|
| ATCC21831 | 0.0 |
| ATCC21831/pLIB_pBL1 | 0.0 |
| ATCC21831_DlysEG/pLIB_pBL1 | 0.0 |
| ATCC21831/pLIB_pBL1_AGAT-Mp | 5.0 |
| ATCC21831_DlysEG/pLIB_pBL1_AGAT-Mp | 5.6 |

As shown in Table 9, ATCC21831, ATCC21831/pLIB_pBL1 and ATCC21831_DlysEG/pLIB_pBL1 are not able to produce GAA.

Strain ATCC21831/pLIB_pBL1_AGAT-Mp also carries a polynucleotide coding for the AGAT from Moorea producens and produces 5.0 g/l GAA. Furthermore, as shown in Table 8, the strain ATCC13032/pLIB_pBL1_AGAT-Mp that carries a polynucleotide coding for the AGAT from Moorea producens, but that is not capable to provide additional L-arginine, only produces 1.1 g/L of GAA.

Strain ATCC21831_DlysEG/pLIB_pBL1_AGAT-Mp also carries a polynucleotide coding for the AGAT from Moorea producens, but additionally the genes lysEG are inactivated in this strain and they have reduced L-arginine export activity. This strain produces 5.6 g/l of GAA, which is improved as compared to the strain without reduced L-arginine export.

We conclude that the combination of enzymatic AGAT activity, increased ability to provide L-arginine and reducing L-arginine export improves GAA production.

### Overview Sequences

SEQ ID NO: 1: Shows the DNA sequence coding for the plasmide pK19mobsacB-DlysEG which was constructed as described in Vrljic et. al 1996 (Vrljic, M., et al. (1996). “A new type of transporter with a new type of cellular function: L-lysine export from Corynebacterium glutamicum.” Mol Microbiol 22(5): 815-826)
SEQ ID NO: 2: Shows the DNA sequence coding for the primer DargR_If
SEQ ID NO: 3: Shows the DNA sequence coding for the primer DargR_Ir
SEQ ID NO: 4: Shows the DNA sequence coding for the plasmid DargR_rf
SEQ ID NO: 5: Shows the DNA sequence coding for the primer DargR_rr
SEQ ID NO: 6: Shows the DNA sequence coding for the primer PsodcarAB-LA-F
SEQ ID NO: 7: Shows the DNA sequence coding for the primer PsodcarAB-LA-R
SEQ ID NO: 8: Shows the DNA sequence coding for the primer PsodcarAB-F
SEQ ID NO: 9: Shows the DNA sequence coding for the primer PsodcarAB-R
SEQ ID NO: 10: Shows the DNA sequence coding for the primer PsodcarAB-RA-F
SEQ ID NO: 11: Shows the DNA sequence coding for the primer PsodcarAB-RA-R
SEQ ID NO: 12: Shows the open reading frame coding for a L-arginine:glycine amidinotransferase (AGAT, EC 2.1.4.1; locus_tag BJP34_00300)
SEQ ID NO: 13: Shows the amino acid sequence derived from SEQ ID No: 12 (Genbank accession Number WP_070390602)
SEQ ID NO: 14: Shows the DNA sequence coding for a L-arginine:glycine amidinotransferase from Moorea producens (AGAT, EC 2.1.4.1) optimized for the codon usage of C. glutamicum.
SEQ ID NO: 15: Shows the DNA sequence coding for a L-arginine:glycine amidinotransferase from Moorea producens (AGAT, EC 2.1.4.1) optimized for the codon usage of C. glutamicum derived from SEQ ID No: 14 with a segment, consisting of base pairs 13-1142, expanded with a BsmBI restriction site at its 5′-end. Furthermore, at the 3′-end a second stop-codon, the lysS-terminator from C. glutamicum and a BsmBI restriction site were added.
SEQ ID NO: 16: DNA segment consisting of a sequence for assembly cloning, a promoter sequence, a ribosomal binding site and the first 81 nucleotides of the optimized AGAT-Mp gene.
SEQ ID NO: 17: Shows the DNA sequence of the shuttle plasmid pLIB_pBL1 has the replication origin from pBL1, the pSC101 replication origin, a kanamycin resistance gene and the BioBricks Terminator BBa_B1006 downstream of a Notl restriction site
SEQ ID NO: 18: Shows the DNA sequence of the primer AGAT_f
SEQ ID NO: 19: Shows the DNA sequence of the primer AGAT_r
SEQ ID NO: 20 Shows the DNA sequence of promotor Pg3-argFGH
SEQ ID NO: 21 Shows the DNA sequence of the primer argFGH_f
SEQ ID NO: 22 Shows the DNA sequence of the primer argFGH_r
SEQ ID NO: 23 Shows the DNA sequence of the plasmid pK18-IBcg0054::Pg3-argFGH

1. A microorganism having an increased ability to provide L-arginine compared with the ability of the wildtype microorganism, comprising:
at least one gene coding for a protein having the function of an L-arginine:glycine amidinotransferase and having a decreased activity of a protein having the function of an arginine exporter compared with the activity of the respective protein in the wildtype microorganism at the same status of the cell cycle.

2. The microorganism of claim 1, wherein the activity of the arginine responsive repressor protein ArgR is attenuated or deleted.

3. The microorganism of claim 1 having increased activities of an enzyme having the function of a carbamoyl phosphate synthase compared to the respective enzymic activity in the wildtype microorganism.

4. The microorganism of claim 3, wherein the increased activity of the enzyme having the function of a carbamoyl phosphate synthase is achieved by a mutation and/or overexpression of a gene coding for the enzyme having the function of a carbamoyl phosphate synthase.

5. The microorganism of claim 1, wherein at least one or more of the genes coding for an enzyme of the biosynthetic pathway of L-omithine and L-arginine, comprising
argF/argF2 coding for an ornithine carbamoyl transferase,
argG coding for an argininosuccinate synthetase and
argH coding for an argininosuccinate lyase
is overexpressed.

6. The microorganism of claim 1, wherein a gene gdh coding for a glutamate dehydrogenase, is overexpressed.

7. The microorganism of claim 1, wherein the gene coding for the protein having the function of an L-arginine:glycine amidinotransferase is heterologous.

8. The microorganism of claim 1, wherein the protein having the function of an L-arginine:glycine amidinotransferase comprises an amino acid sequence which is at least 70% identical to the amino acid sequence according to SEQ ID NO: 13.

9. The microorganism of claim 1, wherein the gene coding for the protein having the function of an arginine exporter is inactivated or deleted.

10. The microorganism of claim 1, wherein a gene coding for a transcriptional activator of the gene coding for the protein having the function of an arginine exporter is deleted.

11. The microorganism of claim 1, wherein the microorganism belongs to the genus Corynebacterium, to the genus Enterobacteriaceae or to the genus Pseudomonas.

12. The microorganism of claim 11, wherein the microorganism is Corynebacterium glutamicum and the gene coding for the protein having the function of an arginine exporter is lysE and the gene coding for the transcriptional activator is lysG.

13. The microorganism of claim 11 wherein the microorganism is Escherichia coli and the gene coding for the protein having the function of an arginine exporter is argO (ybjE).

14. The microorganism of claim 11 wherein the microorganism is Pseudomonas putida and the protein having the function of an arginine exporter is lysE.

15. The method for the fermentative production of guanidino acetic acid (GAA), comprising:
a) cultivating the microorganism as defined in claim 1 in a suitable medium under suitable conditions, and
b) accumulating GAA in the medium to form a GAA containing fermentation broth.

16. The method of claim 15, further comprising:
isolating GAA from the GAA containing fermentation broth.

17. The method of claim 15, further comprising:
drying and/or granulating the GAA containing fermentation broth.

18. The microorganism as claimed in claim 1, further comprising:
a gene coding for an enzyme having the activity of a guanidinoacetate N-methyltransferase.

19. The microorganism of claim 18, wherein the gene coding for an enzyme having the activity of a guanidinoacetate N-methyltransferase is overexpressed.

20. The method for the fermentative production of creatine, comprising:
a) cultivating the microorganism as defined in claim 18 in a suitable medium under suitable conditions, and
b) accumulating creatine in the medium to form a creatine containing fermentation broth.

21. The method of claim 20, further comprising isolating creatine from the creatine containing fermentation broth.


SEQUENCE LISTING (23 sequences)

SEQ ID NO: 1 - DNA, 6544 bp, Artificial Sequence
  misc_feature (1)..(6544): pK19mobsacB-DlysEG
  plasmid constructed as described for pK18mobsacB-lysEG in Vrljic et. al 1996
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccatttgctgaaggtgttactctgcctggcccaattcctgcgggcgaagaagtgaaaaaccctgaaccttttcagaagtaactaaggccgcaatccctcgattgctgcatcaacgacggcgtctgtgagtctagctagagatctagattccaggcgccatcgttgccaatacatcggtgtgtcaatgggtatctcatcgaggaggatcacttctcctgcttttagcatgggagcagcttgggtttcgggaagaagtccccaaccaaggcgcgtttgtgtttatcggcggcgtcggcgcgcaatacggcgacaccggacggtggattttcgccgctggcgcgttcgcggcaagcctgatctggttcccgctggtgggtttcggcgcagcagcattgtcacgcccgctgtccagccccaaggtgtggcgctggatcaacgtcgtcgtggcagttgtgatgaccgcattggccatcaaactgatgttgatgggttagttttcgcgggttttggaatcggtggccttcgcccaaatgttgatgccggcgtcgtgggaaatctcatcgatcgcctccaactcggcgtcagaaaactccaagttgttgagtgaatcaaggctgttgtccagctgctcaactgacgaagcaccaatcaatgcactggtcacggtatccgcgccgtactctccttgctcgcgcagcacccatgcaagcgccatctgcgcaagtgactgcccgcgttcctgggcgatgtcattgagcttgcggaccatatcaatattgttcacgttcaacatgccctcagacagggacttaccctggctggcgcgggaaccctctggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcg

SEQ ID NO: 2 - DNA, 44 bp, Artificial Sequence
  misc_feature (1)..(44): DargR_lf
  DNA sequence coding for the primer DargR_lf (genomic DNA of ATCC13032 as template)
tcgagctcggtacccggggatcctctggctgatgatgatctcac

SEQ ID NO: 3 - DNA, 45 bp, Artificial Sequence
  misc_feature (1)..(45): DargR_lr
  DNA sequence coding for the primer DargR_lr (genomic DNA of ATCC13032 as template)
ctagggatagtagacacttcacaagtgtcttacctcggctggttg

SEQ ID NO: 4 - DNA, 47 bp, Artificial Sequence
  misc_feature (1)..(47): DargR_rf
  DNA sequence coding for the plasmid DargR_rf (genomic DNA of ATCC13032 as template)
cttgtgaagtgtctactatccctagaattactcagcgggcgcaccac

SEQ ID NO: 5 - DNA, 46 bp, Artificial Sequence
  misc_feature (1)..(46): DargR_rr
  DNA sequence coding for the primer DargR_rr (genomic DNA of ATCC13032 as template)
agcttgcatgcctgcaggtcgactctgcacgacggttcagctcttc

SEQ ID NO: 6 - DNA, 46 bp, Artificial Sequence
  misc_feature (1)..(46): PsodcarAB-LA-F
  DNA sequence coding for the primer PsodcarAB-LA-F (genomic DNA of ATCC13032 as template)
ggaaacagctatgacatgattacgcggttatcgcggaatccgtatg

SEQ ID NO: 7 - DNA, 25 bp, Artificial Sequence
  misc_feature (1)..(25): PsodcarAB-LA-R
  DNA sequence coding for the primer PsodcarAB-LA-R (genomic DNA of ATCC13032 as template)
ttaagcgttttgtgcaactccgtct

SEQ ID NO: 8 - DNA, 50 bp, Artificial Sequence
  misc_feature (1)..(50): PsodcarAB-F
  DNA sequence coding for the primer PsodcarAB-F (genomic DNA of ATCC13032 as template)
agacggagttgcacaaaacgcttaaaccctacttagctgccaattattcc

SEQ ID NO: 9 - DNA, 50 bp, Artificial Sequence
  misc_feature (1)..(50): PsodcarAB-R
  DNA sequence coding for the primer PsodcarAB-R (genomic DNA of ATCC13032 as template)
ggtaggtggtggtgtctttactcatgggtaaaaaatcctttcgtaggttt

SEQ ID NO: 10 - DNA, 25 bp, Artificial Sequence
  misc_feature (1)..(25): PsodcarAB-RA-F
  DNA sequence coding for the primer PsodcarAB-RA-F (genomic DNA of ATCC13032 as template)
atgagtaaagacaccaccacctacc

SEQ ID NO: 11 - DNA, 46 bp, Artificial Sequence
  misc_feature (1)..(46): PsodcarAB-RA-R
  DNA sequence coding for the primer PsodcarAB-RA-R (genomic DNA of ATCC13032 as template)
gttgtaaaacgacggccagtgccaccggtgatgtggttcttcactg

SEQ ID NO: 12 - DNA, 1146 bp, Moorea producens
  misc_feature (1)..(1146): L-arginineglycine amidinotransferase
atgtcggaaaaaattgttaattcctggaatgaatgggatgaattggaagaaatggtggtaggaattgcagactatgctagctttgaaccaaaagaaccagggaatcatccgaaattaagaaatcaaaatttagcggaaatcattcctttccccagtggacctaaagaccctaaagtccttgaaaaagctaatgaagaattaaatggactggcttatttattaaaagaccacgatgtgatagtaagaagacccgaaaaaattgattttactaaatctctaaaaacaccttactttgaagttgcaaatcaatactgtggagtctgtcctcgggatgtcatgattacctttgggaatgaaatcatggaagcgactatgtcgaagagagctagattttttgaatacttaccttaccggaaattggtctatgaatattggaataaagacgagcatatgatttggaatgctgcgcctaaaccgactatgcaggatagtatgtatctagagaatttctgggagctgtctttagaagaacgatttaagcgtatgcatgattttgaattttgtattacacaagatgaagtaatttttgatgcggctgactgtagcagattaggaaaggatatattagttcaggaatcgatgacaacaaatagaacaggaattcggtggttaaaaaagcacctagaaccaagaggatttcgggttcaccctgttcattttccccttgattttttcccctcacacattgactgtacgtttgttcctttgcgaccaggtcttattttgacaaaccctgaaagacctatacgggaagaggaggagaagatttttaaagagaatggctgggagttgatcacagttcctcaaccgacttgctcgaatgatgaaatgccaatgttttgccagtccagtaagtggttgtcaatgaatgttctgagtatatcaccgacaaaggttatctgtgaggaaagagaaaaacctctccaagaattgttggataagcatggatttgaggtttttcctttaccctttagacatgtctttgaatttggggggtcttttcattgtgcaacttgggatattcgccgaaaaggtgagtgtgaagattatttaccaaatttaaactatcaaccgatttgtggttaa

SEQ ID NO: 13 - protein, 381 aa, Moorea producens
  misc_feature (1)..(381): L-arginineglycine amidinotransferase
MSEKIVNSWNEWDELEEMVVGIADYASFEPKEPGNHPKLRNQNLAEIIPFPSGPKDPKVLEKANEELNGLAYLLKDHDVIVRRPEKIDFTKSLKTPYFEVANQYCGVCPRDVMITFGNEIMEATMSKRARFFEYLPYRKLVYEYWNKDEHMIWNAAPKPTMQDSMYLENFWELSLEERFKRMHDFEFCITQDEVIFDAADCSRLGKDILVQESMTTNRTGIRWLKKHLEPRGFRVHPVHFPLDFFPSHIDCTFVPLRPGLILTNPERPIREEEEKIFKENGWELITVPQPTCSNDEMPMFCQSSKWLSMNVLSISPTKVICEEREKPLQELLDKHGFEVFPLPFRHVFEFGGSFHCATWDIRRKGECEDYLPNLNYQPICG

SEQ ID NO: 14 - DNA, 1146 bp, Moorea producens
  misc_feature (1)..(1146): L-arginineglycine amidinotransferase codon optimized for C. glutamicum
atgtcagaaaagattgttaactcctggaacgaatgggacgaacttgaagaaatggtggttggcatcgcggactacgcttccttcgagccaaaagaaccaggtaaccaccctaagctacgcaaccagaacctggccgagatcatcccattcccatccggcccaaaggacccaaaggttctcgaaaaagcaaacgaagagctgaacggcctcgcatacctcctaaaggaccacgatgttatcgtccgccgcccagaaaaaattgatttcactaagtcccttaaaaccccatacttcgaggtcgctaaccagtactgcggcgtttgcccacgcgacgttatgattaccttcggtaatgagattatggaggctaccatgagcaagcgtgcacgcttcttcgagtacttgccataccgtaagctcgtctacgagtactggaacaaggatgagcatatgatctggaacgctgctccaaagcctacgatgcaggactccatgtacttggagaacttctgggagctgtccctcgaggagcgcttcaagcgcatgcacgacttcgaattttgcattacccaggacgaggttatcttcgacgcagctgactgctcccgcctgggcaaggacatcctggtccaggagtccatgaccaccaaccgcaccggcattcgttggttgaagaagcacctcgaacctcgcggtttccgcgttcacccagtccacttccctctcgacttcttcccttctcacattgactgcactttcgtccctctccgtcccggcctcatccttaccaacccagagcgcccaatccgcgaagaagaagaaaagatcttcaaggagaacggctgggagcttatcaccgtcccacaacctacctgctccaacgacgaaatgcctatgttctgccagtcctcgaagtggctcagcatgaacgtcctttctatctccccaacaaaggttatttgcgaagagcgtgaaaagccactacaggaactcctggacaagcacggcttcgaagtcttcccactcccattccgccacgttttcgaattcggcggctcctttcactgcgccacttgggacatccgccgcaagggcgaatgcgaagactacctcccaaacctgaactaccagcctatctgcggctaa

SEQ ID NO: 15 - DNA, 1264 bp, Moorea producens
  misc_feature (1)..(1264): L-arginineglycine amidinotransferase segment
agcgtccgtctcctcctggaacgaatgggacgaacttgaagaaatggtggttggcatcgcggactacgcttccttcgagccaaaagaaccaggtaaccaccctaagctacgcaaccagaacctggccgagatcatcccattcccatccggcccaaaggacccaaaggttctcgaaaaagcaaacgaagagctgaacggcctcgcatacctcctaaaggaccacgatgttatcgtccgccgcccagaaaaaattgatttcactaagtcccttaaaaccccatacttcgaggtcgctaaccagtactgcggcgtttgcccacgcgacgttatgattaccttcggtaatgagattatggaggctaccatgagcaagcgtgcacgcttcttcgagtacttgccataccgtaagctcgtctacgagtactggaacaaggatgagcatatgatctggaacgctgctccaaagcctacgatgcaggactccatgtacttggagaacttctgggagctgtccctcgaggagcgcttcaagcgcatgcacgacttcgaattttgcattacccaggacgaggttatcttcgacgcagctgactgctcccgcctgggcaaggacatcctggtccaggagtccatgaccaccaaccgcaccggcattcgttggttgaagaagcacctcgaacctcgcggtttccgcgttcacccagtccacttccctctcgacttcttcccttctcacattgactgcactttcgtccctctccgtcccggcctcatccttaccaacccagagcgcccaatccgcgaagaagaagaaaagatcttcaaggagaacggctgggagcttatcaccgtcccacaacctacctgctccaacgacgaaatgcctatgttctgccagtcctcgaagtggctcagcatgaacgtcctttctatctccccaacaaaggttatttgcgaagagcgtgaaaagccactacaggaactcctggacaagcacggcttcgaagtcttcccactcccattccgccacgttttcgaattcggcggctcctttcactgcgccacttgggacatccgccgcaagggcgaatgcgaagactacctcccaaacctgaactaccagcctatctgcggctaataaggcgcgccttttgaatcctccaaacctgtggatagaacaatctatccacaggttttcagcctgcaggcatgcaagcttggctgttttggcggatgagagaagattttcagcgagacgatcatg

SEQ ID NO: 16 - DNA, 351 bp, Moorea producens
  misc_feature (1)..(351): L-arginineglycine amidinotransferase promoter containing segment
ccgagtccaatgtgtccgtgttcacctcagcggccgcaccggtcggggtaccttgacaaatactttccaatggggtacaatggcgcagtgccatccactccgaggttgataccagcctcctcggtggaatgatcatccgcgtcggagacgaagtaattgacggcagcacctcgggcaaactcgagcgtctgcgggcaagcttcgcataaagacacgacgaattagacaacattagtaatgctggaagaaacaaccgagagcaggaagaacatgtcagaaaagattgttaactcctggaacgaatgggacgaacttgaagaaatggtggttggcatcgcggactacgcttcc

SEQ ID NO: 17 - DNA, 6903 bp, Artificial Sequence
  misc_feature (1)..(6903): pLIB_pBL1
  E. coli-C. glutamicum shuttle plasmid pLIB_pBL 1 has the replication origin from pBL 1, the pSC101 replication origin, a kanamycin resistance gene and the BioBricks Terminator BBa_B1006 downstream of a Natl restriction site
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcctgcagggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatgggcctgcaggcctcagcgcgattcaggcctggtatgagtcagcaacaccttcttcacgaggcagatctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatccgagtccaatgtgtccgtgttcacctcagcggccgcgttaacttaaggtaccgaattctaagcttcacaaaaaaaaaccccgcccctgacagggcggggttttttttactcagcatgtggatggccagtggtcggaccgagcgctttaattaattagcatggtgacacaagcacagtat

SEQ ID NO: 18 - DNA, 20 bp, Artificial Sequence
  misc_feature (1)..(20): AGAT_f
  primer AGAT_f
tggttggcatcgcggactac

SEQ ID NO: 19 - DNA, 57 bp, Artificial Sequence
  misc_feature (1)..(57): AGAT_r
  primer AGAT_r
gcttagaattcggtaccttaagttaacgcggccgcttattagccgcagataggctgg

SEQ ID NO: 20 - DNA, 6026 bp, Corynebacterium glutamicum
  misc_feature (1)..(6026): Pg3-argFGH
ttccggctcgtatgttgtgtggaattgtgagcggataacaatttcacacaggaaacagctatgaccatgattacgaattgcgtggcaatcaggctttacctcagcgaagaattcgacgacgccacccgcgtctacaccgtccgatcctttgatgccgcaactgaaagcatcgtggtggatgtggttcaacaccaccacgaaagccccatgatgcgctggtcagacaccgtaaaaatcaacgacaccctcgtgctcaccggaccccgcccacactttgtcatccccgaaggcgaacaagcagcactcttccttgatgacaccgccatccccgctctcgccgctattttggatcaatggccaacagatcttcgtggcaaaggatgggttgtcactgacgatcccgcagccttcgatgaactacccagcatcgacggactggaactgaacctgctcgcgccgggatcagatccaactgttcagccacttgcccaacaggcatatgacctggaaaacccagaaacttacgtggtgtgggcagccggcgagcgagatgaaataaaatccatccgcaggcacttccgcaagcaggtgggattggaaaaagatgcagtggccgtgtttgggtactggaaatacaacaccaccaacactcagatcgatgcagtccgcaaagaaaactacatgaagatgctctctgaagggctacagctggaaaacttcgacgacctctcattggagatttaaggggtcgagtttttagaatgggtgtttgcattgattggcgtctgatgagctcttagaagcgattctgggagggcatgatttttgggattcggagctgaatgtggactgattttgggggaaccagactggcgtgcccaaatgtcgtgtcttcagtcctaggtcaatattacaggccataaactaatgcttatcgacgtacccccgcacgtgcccactccgcgtgagcggtacttgcggcatcgacacgtgcgcgttgctccgcaacccgcacgcccgcggttccaccgcgcgttgcacgggaagccactgcaccatcaatggtgagcacttcccgtacctctggggtcagacgtgcatcaacgccactgagttcttcatcagtgagatcgataaggtccactcccctggactccgcgatccgcacgcaagcgccggatgcttcgtgtgcctcacggaatggaacgccctggcgcaccatccactcagccaagtcggtggcaagggtgaaacctgctggtgcaagttcacgcatgcgctcggtgttgaaggtcaaggtggaaaccaaaccagtcattgcagggagcagcaggttgagctgcgccacggaatctacgattggttccttatcttcctgcaggtcgcggttgtacgctaaaggctgtgccttcagggtagccagcagaccggtgaggttaccgatcaagcgaccagacttgccacgggtcagctctgccacgtcagggttcttcttctgcggcatgattgagctgcctgtggaccaggaatcagacaaggtgatgtaaccaaattctggggtgcaccatgcgatgatttcttcagccaagcgggacatatccactgcaagctgcgccagcacgaaggcggtttcagatgcgaaatcgcgggagctggtggcatcaatggagttatctgctgcggaatcaaagccgagttcttcagcgattgcttcagggttgagcttcaaagaggaaccagcaagtgcgccggaaccgtaaggagacaccgcaagacgcttgtccaggtcacggatacgatcaatatcgcgcagcaaaggctgtgcgtgtgccagcagctggtgtgccagaaggaccggctgagctgcctggaagtgggtcttgcctggcatgatcgcgcctgcatgtgccttagcttgggcgctgagggcgtcgacaagctcggttgttcccagcgcgatgtcgcgcactgcgtcgcggacccacatgcggaacagggttgccacctggtcgttgcgggaacgaccagcgcgcagacggccgcccacctcaggaccaacgcggtcaatcagaccgcgttccatcgcgccgtgcacatcctcatcagaaggcagcggaccgaaggttccgtcggcgacatccttgcccagctgatcaagcccagccagcatggtggctagatcttcatcagaaagtagatctgcttggtgcaaaaccttggcgtgtgccttggaggccaacacatcataaggggccaaaacccagtcgaaatgagtggagacactcaaggcgaacatggcctcggagggtccaccggagaagcggccgccccacagcgcaccttcattggttccgtgctgttccatgtggtgttcttctactttctaaatacttgaagttctagtctggatgcttgaaaaggtggcttagttgttgccagcttcgcgatcgcgcttgttagcgatcttggaggacagaccgtgcagctggacaaagcccttagccagggtctggtcgaaggtgtcgccggtgtcgtaggtagccaggttgaagtcgtacagggagtggctggaacgacgaccattgatggtgatggaacctgcgtgcagaaccatgcggatatcgccggtgacgtgctcctgggtggaatcaatgaacgcgtccagggagcgcttcagaggtccgaaccacaggccgtcgtatacttcctcagcccaacgtgcgtcaacgccgcgcttgtagcgagccagttcgcgctcgatggtgacatcttccaaagcctcgtgagccttaatcagtgcgattgcgcctggtgcttcgtagatttcgcgggacttgatgcccacgagacggtcctcaaccatgtcaaggcggccaacgccctgtgcgcctgcacgacggttcagctcttcaatagcctgcagtacggagactggacggccatcgatggagactggcttgccaccctcgaaggagatgatgacctcatctggagcgttacccagagctggatcctcggtgtatgcgtagatgtccttggttggagcattccacagatcttccaggtaaccggtctcaatagcgcggccccagacgttctggtcgatggagaatggggacttcacggactgctcgattggaacgttgttctcctcggcgaaggcgatagccttgtcgcgggtccatgcgaagtcacgagcaggtgcaatgatctccaggtttggatcggtgtccatgaagccgacctcgaaacgaacctggtcgttgcccttaccagtgcagccgtgtgcaacgtgggtaccgttgaactgcttgccagcctcaacgaggtgcttgacgatcagtgggcgggagattgcagaaaccagtgggtactgcttcatgtacatgccgtttgccttgatggttggcaggcagtactcctcagcgaactcatcctttgcatcaacaacgatggactcagctgcaccggcatccaatgcacgctggcgaacgttgtccatgttctctccaccctggcccaggtcgagggaaactgcgatgacttcaccatcaatcatcttcttcaggtatggaattgccacagtggtgtccagaccgccggagtatgcaagaacgatgcggttagtcatgaggtgtgctccttcattttaagttccttgccagccgtgttattcttacctcggctggttggccagcagccacaccagcagtgctttctgagcgtggaggcggttttctgcttcatcgaaaactttggacgctggtccatcaatcacggaggctgccacttctttgccacggtaggcaggaaggcagtgcaggaagatggcgccgtcgttagctttcgccatgacctcatcgttgacctggtaaggaacgaaaggtgtggtgcgatcgatgccgtcgttttccatacccatggatacccaggtatcggtgatgacaacatcggcgccggcaacctcgtcgaggctgtcggtgacaacaaccttcgcgccggtttcctggccacgcttttccgcgcgctccacgaattccgcacgaggctggaacccttcaggagcgatgatggaaatatccatgcccgcggtggcaaagccaatcatgtaggagttggccatgttgttgtcgccatcgcccaggtacacagccttcttacccttaaggcctgctgggccttcttcagggctgaggttttccacgatagtctgcagatcagccagaatctggcatgggtgcagatcatcggacaaggagttcaccagcggcacagtggacgtctccgccatggcgtggaaattgctgtgtgcgtaggtgcgccacacaattgcttccacgtagcgggacaatacagctgcggtgtcctgcagggactcgcccttacccatctgtgagctaccggaatccacgacgatggcgtgtccacccaaatgagcgatgcccgcgtcgaaggagaagcgagtacgagttgaagtcttatcaaaaagaactgcaacggactttggtccctcgagtggacgctccgaaaacggcgctgccttgagctttgcggctagggtcaaaacctctgcctgctctgcaggggtgagatcatcatcagccagaaaatggcgaacctgtggttgtgaagtcataagtttgagtcctttatgcgattgtctcggcagttcgattgtaggaaatgcaatgtgtcaagcagttaggtcaatcagagcatcggtcactcaagaattcttgagctgacagaacccaagggcgagtatggcgaattttttcaactcacgcaccaaacctactcacattggataattcgtcgagtcaactccgtgtcaacctgtcctcaactttagcggggaaacaaagatttcgcagcagatgcaaaatcatacagacacattcaaaaatggacattgaccccaaacgggggcatttccaacacttgcgctaaatagtttttgcagggaacgaacctgaaatcgttcgcactacgatctgctcccccgaatggggttaattatgaactacattacacccccgtgaatccacaaattaaagtctcagatttgaagccaatgtgatgcaactcacactggcttcaatggtaatcccaataggctcggtcgtttttaggcttcaatgcatgcctgtctggtttctcggctctcaaaccgaacaggagtgcccacggctggcaaaaagttcagttcttgggcacgctagtctggttttacctttttgagtgccccgaactgccaaacgagagtgcccatgaggggctaaatctggatccgcccgcacaaaacaactgccgcagcacaacgaatcaacgctgtgctgcggcagtcacttttttgaaggattaacgccaacccatggtcatgagtaggccgatgatcatcagaccgaagccgatgccatagttccatgcaccaagatcagccatgaatgggatctgtgggcccacgaggtagttaatgatcaaccaggctaggccgacgatcatgaaggcaaacatgatgaccttgtaccacattggggttccggcggaattgatcttaaccggggtgcggtttgcgcttgggttgcttgaaaccggtgcggtctcgtttttagttactcttgcctttggcattgttcacaccttcataatttattgtcttggaaaaaccccagcaactggtacttatcttgtttgagcttaacgtatcagttgccgggccagctaccttgacggtttccttgttggctattgcacgagtgcagcgagatcgaattcgaagagtcgcacttgcacttgggcgtctttgcggaagagggttgcaggggttggggattggaatccgattttgttttgatccacgagggctgcggtgtggatggggtcgccgacgatcagggattgatctggggcggtccagccagcagcgcggagggcactgatggcctgttcggtgaagcttggcactggccgtcgttttacaacgtcgtgactgggaaaaccctggcgttacccaacttaatcgc

SEQ ID NO: 21 - DNA, 18 bp, Artificial Sequence
  misc_feature (1)..(18): argFGH_f
  primer argFGH_f
tccggctcgtatgttgtg

SEQ ID NO: 22 - DNA, 21 bp, Artificial Sequence
  misc_feature (1)..(21): argFGH_r
  primer argFGH_r
tggcactggccgtcgttttac

SEQ ID NO: 23 - DNA, 11554 bp, Artificial Sequence
  misc_feature (1)..(11554): pK18_IBcg0054::Pg3-argFGH
  misc_feature (2321)..(2321): n is a, c, g, or t
  plasmid pK18_IBcg0054::Pg3-argFGH
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaancgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcgtggcaatcaggctttacctcagcgaagaattcgacgacgccacccgcgtctacaccgtccgatcctttgatgccgcaactgaaagcatcgtggtggatgtggttcaacaccaccacgaaagccccatgatgcgctggtcagacaccgtaaaaatcaacgacaccctcgtgctcaccggaccccgcccacactttgtcatccccgaaggcgaacaagcagcactcttccttgatgacaccgccatccccgctctcgccgctattttggatcaatggccaacagatcttcgtggcaaaggatgggttgtcactgacgatcccgcagccttcgatgaactacccagcatcgacggactggaactgaacctgctcgcgccgggatcagatccaactgttcagccacttgcccaacaggcatatgacctggaaaacccagaaacttacgtggtgtgggcagccggcgagcgagatgaaataaaatccatccgcaggcacttccgcaagcaggtgggattggaaaaagatgcagtggccgtgtttgggtactggaaatacaacaccaccaacactcagatcgatgcagtccgcaaagaaaactacatgaagatgctctctgaagggctacagctggaaaacttcgacgacctctcattggagatttaaggggtcgagtttttagaatgggtgtttgcattgattggcgtctgatgagctcttagaagcgattctgggagggcatgatttttgggattcggagctgaatgtggactgattttgggggaaccagactggcgtgcccaaatgtcgtgtcttcagtcctaggtcaatattacaggccataaactaatgcttatcgacgtacccccgcacgtgcccactccgcgtgagcggtacttgcggcatcgacacgtgcgcgttgctccgcaacccgcacgcccgcggttccaccgcgcgttgcacgggaagccactgcaccatcaatggtgagcacttcccgtacctctggggtcagacgtgcatcaacgccactgagttcttcatcagtgagatcgataaggtccactcccctggactccgcgatccgcacgcaagcgccggatgcttcgtgtgcctcacggaatggaacgccctggcgcaccatccactcagccaagtcggtggcaagggtgaaacctgctggtgcaagttcacgcatgcgctcggtgttgaaggtcaaggtggaaaccaaaccagtcattgcagggagcagcaggttgagctgcgccacggaatctacgattggttccttatcttcctgcaggtcgcggttgtacgctaaaggctgtgccttcagggtagccagcagaccggtgaggttaccgatcaagcgaccagacttgccacgggtcagctctgccacgtcagggttcttcttctgcggcatgattgagctgcctgtggaccaggaatcagacaaggtgatgtaaccaaattctggggtgcaccatgcgatgatttcttcagccaagcgggacatatccactgcaagctgcgccagcacgaaggcggtttcagatgcgaaatcgcgggagctggtggcatcaatggagttatctgctgcggaatcaaagccgagttcttcagcgattgcttcagggttgagcttcaaagaggaaccagcaagtgcgccggaaccgtaaggagacaccgcaagacgcttgtccaggtcacggatacgatcaatatcgcgcagcaaaggctgtgcgtgtgccagcagctggtgtgccagaaggaccggctgagctgcctggaagtgggtcttgcctggcatgatcgcgcctgcatgtgccttagcttgggcgctgagggcgtcgacaagctcggttgttcccagcgcgatgtcgcgcactgcgtcgcggacccacatgcggaacagggttgccacctggtcgttgcgggaacgaccagcgcgcagacggccgcccacctcaggaccaacgcggtcaatcagaccgcgttccatcgcgccgtgcacatcctcatcagaaggcagcggaccgaaggttccgtcggcgacatccttgcccagctgatcaagcccagccagcatggtggctagatcttcatcagaaagtagatctgcttggtgcaaaaccttggcgtgtgccttggaggccaacacatcataaggggccaaaacccagtcgaaatgagtggagacactcaaggcgaacatggcctcggagggtccaccggagaagcggccgccccacagcgcaccttcattggttccgtgctgttccatgtggtgttcttctactttctaaatacttgaagttctagtctggatgcttgaaaaggtggcttagttgttgccagcttcgcgatcgcgcttgttagcgatcttggaggacagaccgtgcagctggacaaagcccttagccagggtctggtcgaaggtgtcgccggtgtcgtaggtagccaggttgaagtcgtacagggagtggctggaacgacgaccattgatggtgatggaacctgcgtgcagaaccatgcggatatcgccggtgacgtgctcctgggtggaatcaatgaacgcgtccagggagcgcttcagaggtccgaaccacaggccgtcgtatacttcctcagcccaacgtgcgtcaacgccgcgcttgtagcgagccagttcgcgctcgatggtgacatcttccaaagcctcgtgagccttaatcagtgcgattgcgcctggtgcttcgtagatttcgcgggacttgatgcccacgagacggtcctcaaccatgtcaaggcggccaacgccctgtgcgcctgcacgacggttcagctcttcaatagcctgcagtacggagactggacggccatcgatggagactggcttgccaccctcgaaggagatgatgacctcatctggagcgttacccagagctggatcctcggtgtatgcgtagatgtccttggttggagcattccacagatcttccaggtaaccggtctcaatagcgcggccccagacgttctggtcgatggagaatggggacttcacggactgctcgattggaacgttgttctcctcggcgaaggcgatagccttgtcgcgggtccatgcgaagtcacgagcaggtgcaatgatctccaggtttggatcggtgtccatgaagccgacctcgaaacgaacctggtcgttgcccttaccagtgcagccgtgtgcaacgtgggtaccgttgaactgcttgccagcctcaacgaggtgcttgacgatcagtgggcgggagattgcagaaaccagtgggtactgcttcatgtacatgccgtttgccttgatggttggcaggcagtactcctcagcgaactcatcctttgcatcaacaacgatggactcagctgcaccggcatccaatgcacgctggcgaacgttgtccatgttctctccaccctggcccaggtcgagggaaactgcgatgacttcaccatcaatcatcttcttcaggtatggaattgccacagtggtgtccagaccgccggagtatgcaagaacgatgcggttagtcatgaggtgtgctccttcattttaagttccttgccagccgtgttattcttacctcggctggttggccagcagccacaccagcagtgctttctgagcgtggaggcggttttctgcttcatcgaaaactttggacgctggtccatcaatcacggaggctgccacttctttgccacggtaggcaggaaggcagtgcaggaagatggcgccgtcgttagctttcgccatgacctcatcgttgacctggtaaggaacgaaaggtgtggtgcgatcgatgccgtcgttttccatacccatggatacccaggtatcggtgatgacaacatcggcgccggcaacctcgtcgaggctgtcggtgacaacaaccttcgcgccggtttcctggccacgcttttccgcgcgctccacgaattccgcacgaggctggaacccttcaggagcgatgatggaaatatccatgcccgcggtggcaaagccaatcatgtaggagttggccatgttgttgtcgccatcgcccaggtacacagccttcttacccttaaggcctgctgggccttcttcagggctgaggttttccacgatagtctgcagatcagccagaatctggcatgggtgcagatcatcggacaaggagttcaccagcggcacagtggacgtctccgccatggcgtggaaattgctgtgtgcgtaggtgcgccacacaattgcttccacgtagcgggacaatacagctgcggtgtcctgcagggactcgcccttacccatctgtgagctaccggaatccacgacgatggcgtgtccacccaaatgagcgatgcccgcgtcgaaggagaagcgagtacgagttgaagtcttatcaaaaagaactgcaacggactttggtccctcgagtggacgctccgaaaacggcgctgccttgagctttgcggctagggtcaaaacctctgcctgctctgcaggggtgagatcatcatcagccagaaaatggcgaacctgtggttgtgaagtcataagtttgagtcctttatgcgattgtctcggcagttcgattgtaggaaatgcaatgtgtcaagcagttaggtcaatcagagcatcggtcactcaagaattcttgagctgacagaacccaagggcgagtatggcgaattttttcaactcacgcaccaaacctactcacattggataattcgtcgagtcaactccgtgtcaacctgtcctcaactttagcggggaaacaaagatttcgcagcagatgcaaaatcatacagacacattcaaaaatggacattgaccccaaacgggggcatttccaacacttgcgctaaatagtttttgcagggaacgaacctgaaatcgttcgcactacgatctgctcccccgaatggggttaattatgaactacattacacccccgtgaatccacaaattaaagtctcagatttgaagccaatgtgatgcaactcacactggcttcaatggtaatcccaataggctcggtcgtttttaggcttcaatgcatgcctgtctggtttctcggctctcaaaccgaacaggagtgcccacggctggcaaaaagttcagttcttgggcacgctagtctggttttacctttttgagtgccccgaactgccaaacgagagtgcccatgaggggctaaatctggatccgcccgcacaaaacaactgccgcagcacaacgaatcaacgctgtgctgcggcagtcacttttttgaaggattaacgccaacccatggtcatgagtaggccgatgatcatcagaccgaagccgatgccatagttccatgcaccaagatcagccatgaatgggatctgtgggcccacgaggtagttaatgatcaaccaggctaggccgacgatcatgaaggcaaacatgatgaccttgtaccacattggggttccggcggaattgatcttaaccggggtgcggtttgcgcttgggttgcttgaaaccggtgcggtctcgtttttagttactcttgcctttggcattgttcacaccttcataatttattgtcttggaaaaaccccagcaactggtacttatcttgtttgagcttaacgtatcagttgccgggccagctaccttgacggtttccttgttggctattgcacgagtgcagcgagatcgaattcgaagagtcgcacttgcacttgggcgtctttgcggaagagggttgcaggggttggggattggaatccgattttgttttgatccacgagggctgcggtgtggatggggtcgccgacgatcagggattgatctggggcggtccagccagcagcgcggagggcactgatggcctgttcggtgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcg